SEQUENCE LISTING (94 sequences)

SEQ ID NO: 1 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 2 - protein, 495 aa, synthetic construct
KFTIVFPHNQKGNWKNVPSNYHYCPSSSDLNWHNDLIGTAXQVKMPXSHKAIQADGWMCHASKWVTTCDFRWYGPKYITXSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEXELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 3 - protein, 495 aa, synthetic construct
KFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 4 - protein, 495 aa, synthetic construct
KFSIVFPQSQKGNWKNVPSSYHYCPSSSDQNWHNDLLGITMKVKMPXTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEXELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 5 - protein, 497 aa, synthetic construct
QIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGR

SEQ ID NO: 6 - protein, 597 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGGDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMVGMGVSCTVTREDGTN

SEQ ID NO: 7 - protein, 597 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGGDCHAPTYLPAEVDGDVKLSSNLVILPGQDLQYVLATYDTSAVEHAVVYYVYSPSRLSSYFYPFRLPIKGVPIELQVECFTWDQKLWCRHFCVLADSESGGHITHSGMVGMGVSCTVTREDGTN

SEQ ID NO: 8 - protein, 497 aa, synthetic construct
LHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 9 - protein, 569 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 10 - protein, 569 aa, synthetic construct
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 11 - protein, 243 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTSGGGGSGGGGSGGGGSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSS

SEQ ID NO: 12 - protein, 240 aa, synthetic construct
DIVMTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKGGGGSGGGGSGGGGSQVQLLESGAELARPGASVKMSCKASGYTFTRYTMHWVKQRPGQGLEWIGYINPSRGYTNYNQKFKDKATLTTDKSSSTAYMQLSSLTSEDSAVYYCAGYYDDHYCLDYWGQGTLVTVSS

SEQ ID NO: 13 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 14 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 15 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 16 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 17 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 18 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 19 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 20 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 21 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 22 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 23 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 24 - protein, 495 aa, synthetic construct
KFTIVFPHNQKGNWKNVPSNYHYCPSSSDLNWHNDLIGTAIQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 25 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 26 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 27 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 28 - protein, 495 aa, synthetic construct
KFTIVFPHNQKGNWKNVPSNYHYCPSSSDLNWHNDLIGTALQVKMPASHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 29 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 30 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 31 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 32 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 33 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 34 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 35 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 36 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 37 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 38 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 39 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 40 - protein, 495 aa, synthetic construct
KFTIVFPHNQKGNWKNVPSNYHYCPSSSDLNWHNDLIGTAIQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 41 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 42 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 43 - protein, 495 aa, synthetic construct
KFTIVFPHNQKGNWKNVPSNYHYCPSSSDLNWHNDLIGTALQVKMPGSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 44 - protein, 495 aa, synthetic construct; identical to SEQ ID NO: 40

SEQ ID NO: 45 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 46 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 47 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 48 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 49 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 50 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 51 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 52 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 53 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 54 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 55 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 56 - protein, 495 aa, synthetic construct
KFTIVFPHNQKGNWKNVPSNYHYCPSSSDLNWHNDLIGTAIQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 57 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 58 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 59 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 60 - protein, 495 aa, synthetic construct
KFTIVFPHNQKGNWKNVPSNYHYCPSSSDLNWHNDLIGTALQVKMPFSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 61 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 62 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 63 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 64 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 65 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 66 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 67 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 68 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITQSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 69 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 70 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 71 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 72 - protein, 495 aa, synthetic construct
KFTIVFPHNQKGNWKNVPSNYHYCPSSSDLNWHNDLIGTAIQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 73 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 74 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEGELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 75 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEFELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 76 - protein, 495 aa, synthetic construct
KFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEQELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 77 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 78 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEGELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 79 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEFELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 80 - protein, 495 aa, synthetic construct
KFSIVFPQSQKGNWKNVPSSYHYCPSSSDQNWHNDLLGITMKVKMPATHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 81 - protein, 495 aa, synthetic construct
KFSIVFPQSQKGNWKNVPSSYHYCPSSSDQNWHNDLLGITMKVKMPGTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 82 - protein, 495 aa, synthetic construct
KFSIVFPQSQKGNWKNVPSSYHYCPSSSDQNWHNDLLGITMKVKMPGTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEGELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 83 - protein, 495 aa, synthetic construct
KFSIVFPQSQKGNWKNVPSSYHYCPSSSDQNWHNDLLGITMKVKMPGTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEFELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 84 - protein, 495 aa, synthetic construct
KFSIVFPQSQKGNWKNVPSSYHYCPSSSDQNWHNDLLGITMKVKMPGTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 85 - protein, 495 aa, synthetic construct
KFSIVFPQSQKGNWKNVPSSYHYCPSSSDQNWHNDLLGITMKVKMPFTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 86 - protein, 495 aa, synthetic construct
KFSIVFPQSQKGNWKNVPSSYHYCPSSSDQNWHNDLLGITMKVKMPFTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEGELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 87 - protein, 495 aa, synthetic construct
KFSIVFPQSQKGNWKNVPSSYHYCPSSSDQNWHNDLLGITMKVKMPFTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEFELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 88 - protein, 495 aa, synthetic construct
KFSIVFPQSQKGNWKNVPSSYHYCPSSSDQNWHNDLLGITMKVKMPFTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 89 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEAELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 90 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEGELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 91 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEFELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 92 - protein, 495 aa, synthetic construct
KFSIVFPQSQKGNWKNVPSSYHYCPSSSDQNWHNDLLGITMKVKMPQTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTEQELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 93 - protein, 20 aa, synthetic construct
RAEGRGSLLTCGDVEENPGP

SEQ ID NO: 94 - protein, 20 aa, synthetic construct
QCTNYALLKLAGDVESNPGP

(19) United States
(12) Patent Application Publication
(10) Pub. No.: US 2026/0218229 A1
(43) Pub. Date: Jul. 30, 2026

(54) RECOMBINANT RETROVIRUSES, COMPOSITIONS, AND METHODS OF USE

(71) Applicant: Kelonia Therapeutics, Inc., Boston, MA (US)

(72) Inventors: Kevin M. Friedman, Boston, MA (US); Michael J. McDonald, Boston, MA (US)

(21) Appl. No.: 19/143,497

(22) PCT Filed: Dec. 29, 2023
(86) PCT No.: PCT/US2023/086471

§ 371 Date: Jun. 26, 2025

Related U.S. Application Data
(60) Provisional application No. 63/477,816, filed on Dec. 29, 2022.
(60) Provisional application No. 63/453,034, filed on Mar. 17, 2023.

(51) Int. Cl. C12N 15/86; C07K 14/005; C07K 14/715; C07K 16/28; C12N 9/90

(52) U.S. Cl. CPC C12N 15/86; C07K 14/005; C07K 14/7155; C07K 16/28; C07K 16/2803; C07K 16/2809; C07K 16/2878; C07K 16/2887; C07K 16/2896; C12N 9/90; C12Y 502/01008; C12N 2740/15043; C12N 2740/16043; C12N 2760/18222; C12N 2760/18422; C12N 2760/20222

(57) ABSTRACT

The present disclosure provides a recombinant retrovirus comprising retroviral vectors encoding components of a macromolecular complex, which when expressed in a cell and in the presence of a ligand may promote growth and/or survival of the cell.

### CROSS REFERENCE TO RELATED APPLICATIONS

This application claims the benefit under 35 U.S. C. § 119(e) of U.S. Provisional Application No. 63/477,816, filed Dec. 29, 2022, and U.S. Provisional Application No. 63/453,034, filed Mar. 17, 2023, each of which is incorporated by reference herein in its entirety.

### STATEMENT REGARDING SEQUENCE LISTING

The Sequence Listing associated with this application is provided in text format in lieu of a paper copy and is hereby incorporated by reference into the specification. The name of the text file containing the Sequence Listing is KELO-404-WO1_ST26.xml. The text file is 125 KB, was created on Dec. 12, 2023, and is being submitted electronically via EFS-Web, concurrent with the filing of the specification.

### TECHNICAL FIELD

The present disclosure relates to recombinant viruses engineered for transducing cells in vivo. More particularly, the disclosure relates to recombinant viruses comprising viral vectors encoding components of a macromolecular complex, compositions, and in vivo methods.

### DESCRIPTION OF THE RELATED ART

Various vectors suitable for gene therapy are constrained by size limits of the polynucleotides to be delivered. Expensive manufacturing strategies also heavily contribute to the obstacles preventing widespread adoption of this treatment modality. And for systems with larger limits, in vivo delivery has been challenging.

### BRIEF SUMMARY

The present disclosure generally relates, in part, to recombinant retroviruses suitable for in vivo administration to a subject, said retroviruses comprising retroviral vectors encoding components of a macromolecular complex, wherein assembly and activity of the complex is regulated by the presence of a ligand or small molecule.

In various embodiments, a system comprising a recombinant retrovirus is contemplated, said retrovirus comprising: a viral envelope; one or more mutated viral envelope glycoproteins that retain fusogenic activity and lack cognate receptor binding activity, i.e., the ability to bind its cognate receptor expressed on a cell; a non-viral membrane-bound tropism polypeptide, and a recombinant retroviral vector comprising at least a first polynucleotide and a second polynucleotide, each polynucleotide comprising a polynucleotide sequence encoding a polypeptide component of a macromolecular complex, wherein assembly of the macromolecular complex in a cell transduced with the first and second polynucleotides promotes growth and/or survival of a cell. The viral envelope comprises a mutated viral envelope glycoprotein that retains fusogenic activity and lacks cognate receptor binding activity, one or more non-viral membrane-bound tropism polypeptides, and optionally one or more transduction enhancing molecules.

In various embodiments, a system comprising a first recombinant retrovirus and a second recombinant retrovirus is contemplated, comprising: a first recombinant retrovirus comprising a viral envelope, one or more mutated viral envelope glycoproteins that retain fusogenic activity and lack cognate receptor binding activity, a non-viral membrane-bound tropism polypeptide, and a recombinant retroviral vector comprising a first polynucleotide encoding a polypeptide component of a macromolecular complex; and a second recombinant retrovirus comprising a viral envelope; one or more mutated viral envelope glycoproteins that retain fusogenic activity and lack cognate receptor binding activity; a non-viral membrane-bound tropism polypeptide, and a recombinant retroviral vector comprising a second polynucleotide encoding a polypeptide component of a macromolecular complex; wherein assembly of the macromolecular complex in the cell transduced with the first polynucleotide and the second polynucleotide promotes growth and/or survival of a cell. The viral envelopes comprise a mutated viral envelope glycoprotein that retains fusogenic activity and lacks cognate receptor binding activity, one or more non-viral membrane-bound tropism polypeptides, and optionally one or more transduction enhancing molecules.

In particular embodiments, the system comprises a first polynucleotide comprising a polynucleotide sequence encoding a first polypeptide component of the macromolecular complex comprising an FKBP-rapamycin complex binding (FRB) polypeptide or a functional variant thereof, and a second polynucleotide comprising a polynucleotide sequence encoding a second polypeptide component of the macromolecular complex comprising an FK506 binding protein (FKBP) polypeptide or a functional variant thereof; and/or wherein the ligand is rapamycin. In certain embodiments, the first polynucleotide comprises a polynucleotide sequence encoding: a promoter, an FKBP polypeptide, an IL-2 receptor transmembrane domain, an IL2Rγ intracellular signaling domain; and a first chimeric antigen receptor (CAR) and the second polynucleotide comprises a polynucleotide sequence encoding: a promoter, an FRB polypeptide, an IL-2 receptor transmembrane domain, an IL2Rβ intracellular signaling domain; and a second CAR. In particular embodiments, the FKBP polypeptide and FRB polypeptide heterodimerize in the presence of rapamycin to promote growth and/or survival of a cell.

In particular embodiments, a method comprising administering to a subject a system of any of the embodiments contemplated herein is provided.

In various embodiments, a system comprising a recombinant retrovirus comprises (a) a viral envelope comprising (i) one or more mutated viral envelope glycoproteins that retain fusogenic activity and lack cognate receptor binding activity and (ii) a non-viral membrane-bound tropism polypeptide, and (b) a recombinant retroviral vector comprising at least a first polynucleotide and a second polynucleotide, each polynucleotide comprising a polynucleotide sequence encoding a polypeptide component of a macromolecular complex, wherein assembly of the macromolecular complex in a cell transduced with the first and second polynucleotides promotes growth and/or survival of a cell.

In various embodiments, a system comprises (a) a first recombinant retrovirus comprising: (i) a viral envelope comprising one or more mutated viral envelope glycoproteins that retain fusogenic activity and lack cognate receptor binding activity and a non-viral membrane-bound tropism polypeptide, and (ii) a recombinant retroviral vector comprising a first polynucleotide encoding a polypeptide component of a macromolecular complex; and (b) a second recombinant retrovirus comprising: (i) a viral envelope comprising one or more mutated viral envelope glycoproteins that retain fusogenic activity and lack cognate receptor binding activity and a non-viral membrane-bound tropism polypeptide, and (ii) a recombinant retroviral vector comprising a second polynucleotide encoding a polypeptide component of a macromolecular complex; wherein assembly of the macromolecular complex in the cell transduced with the first polynucleotide and the second polynucleotide promotes growth and/or survival of a cell.

In particular embodiments, the macromolecular complex is a multi-component cell-surface receptor.

In further embodiments, the recombinant retrovirus is a recombinant lentivirus.

In some embodiments, the recombinant retroviruses are recombinant lentiviruses.

In additional embodiments, the recombinant retrovirus is a recombinant lentivirus selected from the group consisting of: human immunodeficiency virus 1 (HIV-1); human immunodeficiency virus 2 (HIV-2), visna-maedi virus (VMV); caprine arthritis-encephalitis virus (CAEV); equine infectious anemia virus (EIAV); feline immunodeficiency virus (FIV); bovine immune deficiency virus (BIV); and simian immunodeficiency virus (SIV).

In particular embodiments, assembly of the macromolecular complex is controlled by a ligand.

In further embodiments, the one or more mutated viral envelope glycoproteins comprise a vesiculovirus envelope glycoprotein, one or more morbillivirus envelope glycoproteins or one or more henipavirus envelope glycoproteins.

In certain embodiments, the vesiculovirus is selected from the group consisting of: vesicular stomatitis Alagoas virus (VSAV; Alagoas vesiculovirus), Carajás virus (CJSV; Carajas vesiculovirus), Chandipura virus (CHPV; Chandipura vesiculovirus), Cocal virus (COCV; Cocal vesiculovirus), vesicular stomatitis Indiana virus (VSIV; Indiana vesiculovirus), Isfahan virus (ISFV; Isfahan vesiculovirus), Maraba virus (MARAV; Maraba vesiculovirus), Morreton virus (MORV; Morreton vesiculovirus), vesicular stomatitis New Jersey virus (VSNJV; New Jersey vesiculovirus), and Piry virus (PIRYV; Piry vesiculovirus).

In some embodiments, vesiculovirus envelope glycoprotein is a vesiculovirus G protein.

In particular embodiments, the vesiculovirus G protein is a COCV G glycoprotein (COCV-G) or a VSIV G glycoprotein (VSIV-G).

In certain embodiments, the VSIV-G envelope protein comprises one or more of: (a) one or more amino acid substitutions at H8, N9, Q10, K47, K50, A51, S183, S179, N180, 1182, M184, Y209, 1347, T350, T352, E353, and R354; (b) an insertion of TT between N9 and Q10, an insertion of GGS between H8 and N9, an insertion of GGS between N9 and Q10, an insertion of TT between N208 and Y209, an insertion of GGS between P46 and K47, and an insertion of GGS between N208 and Y209; (c) amino acid substitutions at K47 and/or R354; or (d) a deletion of residues 1-8.

In additional embodiments, the VSIV-G envelope protein comprises one or more amino acid substitutions at H8, K47, Y209, and R354.

In some embodiments, the VSIV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q.

In particular embodiments, the VSIV-G envelope protein comprises the amino acid sequence set forth in SEQ ID NO: 2, wherein X₁=I, X₂=A, X₃=Q, and X₄=A; X₁=I, X₂=A, X₃=Q, and X₄=G; X₁=I, X₂=A, X₃=Q, and X₄=F; X₁=I, X₂=A, X₃=Q, and X₄=Q; X₁=L, X₂=A, X₃=Q, and X₄=A; X₁=L, X₂=A, X₃=Q, and X₄=G; X₁=L, X₂=A, X₃=Q, and X₄=F; X₁=L, X₂=A, X₃=Q, and X₄=Q; X₁=I, X₂=A, X₃=H, and X₄=A; X₁=I, X₂=A, X₃=H, and X₄=G; X₁=I, X₂=A, X₃=H, and X₄=F; X₁=I, X₂=A, X₃=H, and X₄=Q; X₁=L, X₂=A, X₃=H, and X₄=A; X₁=L, X₂=A, X₃=H, and X₄=G; X₁=L, X₂=A, X₃=H, and X₄=F; X₁=L, X₂=A, X₃=H, and X₄=Q; X₁=I, X₂=G, X₃=Q, and X₄=A; X₁=I, X₂=G, X₃=Q, and X₄=G; X₁=I, X₂=G, X₃=Q, and X₄=F; X₁=I, X₂=G, X₃=Q, and X₄=Q; X₁=L, X₂=G, X₃=Q, and X₄=A; X₁=L, X₂=G, X₃=Q, and X₄=G; X₁=L, X₂=G, X₃=Q, and X₄=F; X₁=L, X₂=G, X₃=Q, and X₄=Q; X₁=I, X₂=G, X₃=H, and X₄=A; X₁=I, X₂=G, X₃=H, and X₄=G; X₁=I, X₂=G, X₃=H, and X₄=F; X₁=I, X₂=G, X₃=H, and X₄=Q; X₁=L, X₂=G, X₃=H, and X₄=A; X₁=L, X₂=G, X₃=H, and X₄=G; X₁=L, X₂=G, X₃=H, and X₄=F; X₁=L, X₂=G, X₃=H, and X₄=Q; X₁=I, X₂=F, X₃=Q, and X₄=A; X₁=I, X₂=F, X₃=Q, and X₄=G; X₁=I, X₂=F, X₃=Q, and X₄=F; X₁=I, X₂=F, X₃=Q, and X₄=Q; X₁=L, X₂=F, X₃=Q, and X₄=A; X₁=L, X₂=F, X₃=Q, and X₄=G; X₁=L, X₂=F, X₃=Q, and X₄=F; X₁=L, X₂=F, X₃=Q, and X₄=Q; X₁=I, X₂=F, X₃=H, and X₄=A; X₁=I, X₂=F, X₃=H, and X₄=G; X₁=I, X₂=F, X₃=H, and X₄=F; X₁=I, X₂=F, X₃=H, and X₄=Q; X₁=L, X₂=F, X₃=H, and X₄=A; X₁=L, X₂=F, X₃=H, and X₄=G; X₁=L, X₂=F, X₃=H, and X₄=F; X₁=L, X₂=F, X₃=H, and X₄=Q; X₁=I, X₂=Q, X₃=Q, and X₄=A; X₁=I, X₂=Q, X₃=Q, and X₄=G; X₁=I, X₂=Q, X₃=Q, and X₄=F; X₁=I, X₂=Q, X₃=Q, and X₄=Q; X₁=L, X₂=Q, X₃=Q, and X₄=A; X₁=L, X₂=Q, X₃=Q, and X₄=G; X₁=L, X₂=Q, X₃=Q, and X₄=F; X₁=L, X₂=Q, X₃=Q, and X₄=Q; X₁=I, X₂=Q, X₃=H, and X₄=A; X₁=I, X₂=Q, X₃=H, and X₄=G; X₁=I, X₂=Q, X₃=H, and X₄=F; X₁=I, X₂=Q, X₃=H, and X₄=Q; X₁=L, X₂=Q, X₃=H, and X₄=A; X₁=L, X₂=Q, X₃=H, and X₄=G; X₁=L, X₂=Q, X₃=H, and X₄=F; and X₁=L, X₂=Q, X₃=H, and X₄=Q.

In further embodiments, the vesiculovirus G protein is COCV-G.

In particular embodiments, the COCV-G envelope protein comprises one or more amino acid substitutions at K47 and R354.

In particular embodiments, the COCV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q.

In additional embodiments, the COCV-G envelope protein comprises the amino acid sequence set forth in SEQ ID NO: 4, wherein X₁=A and X₂=A; X₁=A and X₂=G; X₁=A and X₂=F; X₁=A and X₂=Q; X₁=G and X₂=A; X₁=G and X₂=G; X₁=G and X₂=F; X₁=G and X₂=Q; X₁=F and X₂=A; X₁=F and X₂=G; X₁=F and X₂=F; X₁=F and X₂=Q; X₁=Q and X₂=A; X₁=Q and X₂=G; X₁=Q and X₂=F; or X₁=Q and X₂=Q.

In certain embodiments, the one or more morbillivirus envelope glycoproteins are measles virus F (MV-F) and measles virus H (MV-H).

In further embodiments, the MV-H protein comprises one or more amino acid substitutions at Y481, R533, S548, and F549.

In some embodiments, the MV-H protein comprises one or more amino acid substitutions selected from the group consisting of: Y481A, R533A, S548L, and F549S.

In particular embodiments, the one or more henipavirus envelope glycoproteins are nipah virus F (NiV-F) and nipah virus G (NiV-G).

In certain embodiments, the NiV-G protein comprises one or more amino acid substitutions at E501, W504, Q530, and E533.

In additional embodiments, the NiV-G protein comprises one or more amino acid substitutions at E501A, W504A, Q530A, and E533A.

In particular embodiments, the non-viral membrane-bound tropism polypeptide comprises an extracellular antigen targeting domain, a spacer polypeptide, and a transmembrane domain.

In further embodiments, the extracellular antigen targeting domain binds an antigen expressed on an immune effector cell.

In particular embodiments, the extracellular antigen targeting domain binds an antigen expressed on an immune effector cell selected from the group consisting of: the alpha, beta, gamma, or delta chain of the T cell receptor, CD2, CD3δ, CD3εCD3γ, CD4, CD8α, and CD8β.

In certain embodiments, the extracellular antigen targeting domain comprises an antibody or antigen binding fragment thereof that binds an antigen expressed on the immune effector cell.

In some embodiments, the extracellular antigen targeting domain comprises an anti-CD3 antibody or antigen binding fragment selected from the group consisting of OKT3, UCHT1, YTH12.5, TR66, and variants thereof, and antibodies and antigen binding fragments that have at least 80%, at least 85%, at least 90%, at least 95%, at least 99%, or 100% identity thereto.

In particular embodiments, the system comprises a first polynucleotide comprising a polynucleotide sequence encoding a first polypeptide component of the macromolecular complex comprising an FKBP-rapamycin complex binding (FRB) polypeptide or a functional variant thereof, and a second polynucleotide comprising a polynucleotide sequence encoding a second polypeptide component of the macromolecular complex comprising an FK506 binding protein (FKBP) polypeptide or a functional variant thereof; and/or wherein the ligand is rapamycin.

In additional embodiments, expression of the macromolecular complex is under the control of an inducible genetic or biochemical system.

In some embodiments, each polynucleotide is operatively linked to a promoter.

In certain embodiments, the promoter is an inducible promoter.

In additional embodiments, at least one of the polynucleotides comprises a polynucleotide sequence that confers resistance to an immunosuppressive agent.

In further embodiments, the polynucleotide sequence that confers resistance to an immunosuppressive agent encodes a polypeptide that binds rapamycin, wherein optionally, the polypeptide is FRB.

In particular embodiments, the recombinant retrovirus or recombinant retroviruses are capable of transducing T cells, NK cells, or NKT cells in vivo or ex vivo.

In some embodiments, the viral envelope further comprises one or more transduction enhancers, wherein the transduction enhancer is selected from the group consisting of a T cell activation receptor, a NK-cell activation receptor, and a co-stimulatory molecule.

In particular embodiments, the one or more transduction enhancers comprise one or more of CD80, CD86, CD137L, OX40L, and ICOSL.

In certain embodiments, the first polynucleotide comprises a polynucleotide sequence encoding: a promoter, an FKBP polypeptide, an IL-2 receptor transmembrane domain, an IL2Rγ intracellular signaling domain; and a first chimeric antigen receptor (CAR).

In particular embodiments, the second polynucleotide comprises a polynucleotide sequence encoding: a promoter, an FRB polypeptide, an IL-2 receptor transmembrane domain, an IL2Rβ intracellular signaling domain; and a second CAR.

In further embodiments, the FKBP polypeptide and FRB polypeptide heterodimerize in the presence of rapamycin to promote growth and/or survival of a cell. In some embodiments, the promoter is MND.

In particular embodiments, the first CAR is an anti-CD19 CAR, an anti-CD22 CAR, an anti-CD20 CAR, an anti-CD79A CAR, an anti-CD79b CAR, an anti-CD38 CAR, an anti-GPCR5D CAR, or an anti-BCMA CAR.

In certain embodiments, the second CAR is different from the first CAR and is selected from the group consisting of: an anti-CD19 CAR, an anti-CD22 CAR, an anti-CD20 CAR, an anti-CD79A CAR, an anti-CD79b CAR, an anti-CD38 CAR, an anti-GPCR5D CAR, or an anti-BCMA CAR.

In various embodiments, a method comprises administering to a subject a system contemplated herein.

Further aspects and embodiments of the invention are provided by the Detailed Description that follows.

### BRIEF DESCRIPTION OF THE SEQUENCE IDENTIFIERS

SEQ ID NOs: 1-10 set forth amino acid sequences of fusogens.

SEQ ID NOs: 11-12 set forth amino acid sequences of anti-CD3 antibodies.

SEQ ID NOs: 13-92 set forth amino acid sequences of fusogens.

SEQ ID NOs: 93-94 set forth amino acid sequences of viral self-cleaving polypeptides.

In the foregoing sequences, X, if present, refers to any amino acid, a specified group of amino acids or the absence of an amino acid.

Throughout the disclosure, the amino acid position(s) of a fusogen is with reference to the fusogen lacking a signal sequence (i.e., the amino acid sequence after the signal peptide has been cleaved).

### DETAILED DESCRIPTION

### A. Overview

The present disclosure generally relates to, in part, a system comprising one or more recombinant retroviruses suitable for in vivo administration to a subject, said retroviruses comprising retroviral vectors encoding components of a macromolecular signaling complex, wherein assembly and activity of the complex is regulated by the presence of a ligand or small molecule. As used herein, the terms “recombinant retrovirus,” “recombinant retroviral particle” and “retroviral particle” are used synonymously and refer to an enveloped retrovirus comprising an engineered retroviral vector.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 4th ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience (2002); Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid the Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells and Enzymes (IRL Press, 1986); the treatise, Methods in Enzymology (Academic Press, Inc., N.Y.); Gene Transfer Vectors for Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods in Cell and Molecular Biology (Mayer and Walker, eds., Academic Press, London, 1987); Handbook of Experimental Immunology, Volumes I-IV (D. M. Weir and CC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description is for the purpose of describing particular embodiments only and is not intended to be limiting.

As used herein, the singular articles “a”, “an”, and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (or).

Unless the context indicates otherwise, it is specifically intended that the various features described herein can be used in any combination. Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed.

It will also be understood that, as used herein, the terms example, exemplary, and grammatical variations thereof are intended to refer to non-limiting examples and/or variant embodiments discussed herein, and are not intended to indicate preference for one or more embodiments discussed herein compared to one or more other embodiments.

The terms “variant”, “homologue” or “derivative” in relation to a nucleotide sequence include any substitution of, variation of, modification of, replacement of, deletion of or addition of one (or more) nucleic acid from or to the sequence.

### C. Recombinant Retrovirus Systems

The present disclosure provides, in part, a system comprising one or more recombinant retroviruses. In particular embodiments, a system comprises a recombinant retrovirus comprising: a viral envelope; one or more mutated viral envelope glycoproteins modified to retain fusogenic activity and decrease, reduce, substantially ablate, ablate, abolish or eliminate cell binding or attachment activity; a non-viral membrane-bound tropism polypeptide, and a recombinant retroviral vector comprising at least a first polynucleotide and a second polynucleotide, each polynucleotide comprising a polynucleotide sequence encoding a polypeptide component of a macromolecular signaling complex, wherein assembly of the complex in a cell transduced with the first and second polynucleotides promotes growth and/or survival of a cell.

In particular embodiments, a system comprises (a) a first recombinant retrovirus comprising: a viral envelope; one or more mutated viral envelope glycoproteins that retain fusogenic activity and lack cognate receptor binding activity (i.e., modified to retain fusogenic activity and decrease, reduce, substantially ablate, ablate, abolish or eliminate cell binding or attachment activity); a non-viral membrane-bound tropism polypeptide, and a recombinant retroviral vector comprising a first polynucleotide encoding a polypeptide component of a macromolecular signaling complex; and a second recombinant retrovirus comprising: a viral envelope; one or more mutated viral envelope glycoproteins that retain fusogenic activity and lack cognate receptor binding activity (i.e., modified to retain fusogenic activity and decrease, reduce, substantially ablate, ablate, abolish or eliminate cell binding or attachment activity); a non-viral membrane-bound tropism polypeptide, and a recombinant retroviral vector comprising a second polynucleotide encoding a polypeptide component of a macromolecular signaling complex; wherein assembly of the macromolecular signaling complex in the cell transduced with the first polynucleotide and the second polynucleotide promotes growth and/or survival of a cell.

In particular embodiments, a system comprises a macromolecular signaling complex that is a multicomponent cell-surface receptor, optionally a proliferation receptor, optionally a proliferation receptor induced by a ligand.

In particular embodiments, a system comprises a retrovirus comprising a single retroviral vector comprising a first polynucleotide and a second polynucleotide.

In particular embodiments, a system comprises a first retrovirus comprising a first retroviral vector comprising a first polynucleotide and a second retrovirus comprising a second retroviral vector comprising a second polynucleotide. single vector that is a single lentivirus vector.

In preferred embodiments, the retrovirus is a lentivirus.

In preferred embodiments, the first retrovirus and the second retrovirus are both lentiviruses.

In particular embodiments, the assembly of the macromolecular signaling complex is controlled by a ligand, optionally the ligand rapamycin.

In particular embodiments, the ligand is a protein, an antibody, a small molecule, or a drug. In particular embodiments, the ligand is rapamycin or a rapamycin analog (rapalogs). In particular embodiments, the rapalog is everolimus, novolimus, pimecrolimus, ridaforolimus, tacrolimus, temsirolimus, umirolimus, zotarolimus, CCI-779, C20-methallylrapamycin, C16-(S)-3-methylindolerapamycin, C16-iRap, AP21967, sodium mycophemolic acid, benidipine hydrochloride, rapamine, AP23573, or AP1903, or metabolites, derivatives, and/or combinations thereof. In some embodiments, the ligand is an IMID-class drug (e.g. thalidomide, pomalidimide, lenalidomide or related analogues).

In particular embodiments, the ligand is FK1012, tacrolimus (FK506), FKCsA, rapamycin, coumermycin, gibberellin, HaXS, TMP-HTag, and ABT-737 or functional derivatives thereof.

In particular embodiments, the system comprises a first retroviral vector comprising a first polynucleotide comprising a polynucleotide sequence encoding a first polypeptide component of the macromolecular signaling complex comprising an FKBP-rapamycin complex binding (FRB) polypeptide or a functional variant thereof (e.g., FRB T2098L).

In particular embodiments, the system comprises a second retroviral vector comprising a second polynucleotide comprising a polynucleotide sequence encoding a second polypeptide component of the macromolecular signaling complex comprising an FK506 binding protein (FKBP) polypeptide or a functional variant thereof (e.g., FKBP F36V, FKBP 12).

In particular embodiments, the system comprises a retroviral vector comprising a first polynucleotide comprising a polynucleotide sequence encoding a first polypeptide component of the macromolecular signaling complex comprising an FRB polypeptide or a functional variant thereof, and a second polynucleotide comprising a polynucleotide sequence encoding a second polypeptide component of the macromolecular signaling complex comprising an FKBP polypeptide or a functional variant thereof.

In particular embodiments, at least one of the polynucleotides comprises a polynucleotide sequence that confers resistance to an immunosuppressive agent. In certain embodiments, the polynucleotide sequence that confers resistance to an immunosuppressive agent encodes a polypeptide that binds rapamycin, wherein optionally, the polypeptide is FRB.

In particular embodiments, at least one of the polynucleotides comprises a polynucleotide sequence that encodes a cytosolic FRB polypeptide.

In particular embodiments, an FRB polypeptide or a portion thereof and FKBP polypeptide or a portion thereof form a complex that sequesters rapamycin in the transduced cell.

In particular embodiments, an FKBP polypeptide or a portion thereof and FRB polypeptide or a portion thereof heterodimerize in the presence of rapamycin to promote growth and/or survival of a cell.

In one embodiment, FRB T2098L heterodimerizes with FKBP12, in the presence of rapalog AP21967 to promote growth and/or survival of a cell.

In another embodiment, FRB heterodimerizes with FKBP12, in the presence of rapamycin, temsirolimus or everolimus to promote growth and/or survival of a cell.

In another embodiment, FKBP homodimerizes with FKBP in the presence of FK1012 to promote growth and/or survival of a cell.

In another embodiment, FKBP F36V homodimerizes with FKBP F36V in the presence of AP1903 to promote growth and/or survival of a cell.

In particular embodiments, expression and/or activity of the macromolecular signaling complex is under the control of an inducible genetic or biochemical system.

In particular embodiments, one or more polynucleotides is operatively linked to a promoter, optionally an inducible promoter. Illustrative promoters include, without limitation, an EF1α promoter, a cytomegalovirus (CMV) promoter, a CAG promoter, an SV40 promoter, an SV40/CD43 promoter, and an MNDU3 promoter.

In particular embodiments, the promoter is an MNDU3 promoter.

In particular embodiments, a system comprises a vector comprising a first polynucleotide sequence encoding: a promoter, an FKBP polypeptide or a portion thereof, a transmembrane domain, an intracellular signaling domain; and a first CAR.

In particular embodiments, a system comprises a vector comprising a second polynucleotide sequence encoding: a promoter, an FRB polypeptide or a portion thereof, a transmembrane domain, an intracellular signaling domain; and a second CAR.

In particular embodiments, a system comprises a vector comprising a first polynucleotide sequence encoding: a promoter, an FKBP polypeptide or a portion thereof, a transmembrane domain, an IL2Rγ intracellular signaling domain; and a first CAR. In particular embodiments, a system comprises a vector comprising a second polynucleotide sequence encoding: a promoter, an FRB polypeptide or a portion thereof, a transmembrane domain, an IL2Rβ intracellular signaling domain; and a second CAR.

In particular embodiments, the IL2Rγ and IL2Rβ intracellular signaling domains heterodimerize. In particular embodiments, the IL2Rγ and IL2Rβ intracellular signaling domains heterodimerize in the presence of a ligand to promote growth and/or survival of a cell.

In particular embodiments, a system comprises a vector comprising a first polynucleotide sequence encoding: a promoter, an FKBP polypeptide or a portion thereof, a transmembrane domain, an IL2Rγ intracellular signaling domain; and a first CAR.

In particular embodiments, a system comprises a vector comprising a second polynucleotide sequence encoding: a promoter, an FRB polypeptide or a portion thereof, a transmembrane domain, an IL7Rα intracellular signaling domain; and a second CAR.

In particular embodiments, the IL2Rγ and IL7Rα intracellular signaling domains heterodimerize. In particular embodiments, the IL2Rγ and IL7Rα intracellular signaling domains heterodimerize in the presence of a ligand to promote growth and/or survival of a cell.

In particular embodiments, a system comprises a vector comprising a first polynucleotide sequence encoding: a promoter, an FKBP polypeptide or a portion thereof, a transmembrane domain, an IL12Rβ1 intracellular signaling domain; and a first CAR.

In particular embodiments, a system comprises a vector comprising a second polynucleotide sequence encoding: a promoter, an FRB polypeptide or a portion thereof, a transmembrane domain, an IL12Rβ2 intracellular signaling domain; and a second CAR.

In particular embodiments, the IL12Rβ1 and IL12Rβ2 intracellular signaling domains heterodimerize. In particular embodiments, the IL12Rβ1 and IL12Rβ2 intracellular signaling domains heterodimerize in the presence of a ligand to promote growth and/or survival of a cell.

In particular embodiments, a system comprises a vector comprising a first polynucleotide sequence encoding: a promoter, an FKBP polypeptide or a portion thereof, a transmembrane domain, an IL2Rγ intracellular signaling domain; and a first CAR.

In particular embodiments, a system comprises a vector comprising a second polynucleotide sequence encoding: a promoter, an FRB polypeptide or a portion thereof, a transmembrane domain, an IL21R intracellular signaling domain; and a second CAR.

In particular embodiments, the IL2Rγ and IL21R intracellular signaling domains heterodimerize. In particular embodiments, the IL2Rγ and IL21R intracellular signaling domains heterodimerize in the presence of a ligand to promote growth and/or survival of a cell.

In particular embodiments, a system comprises a vector comprising a first polynucleotide sequence encoding: a promoter, an FKBP polypeptide or a portion thereof, a transmembrane domain, an IFNRα1 intracellular signaling domain; and a first CAR. In particular embodiments, a system comprises a vector comprising a second polynucleotide sequence encoding: a promoter, an FRB polypeptide or a portion thereof, a transmembrane domain, an IFNRα2 intracellular signaling domain; and a second CAR.

In particular embodiments, the IFNRα1 and IFNRα2 intracellular signaling domains heterodimerize. In particular embodiments, the IFNRα1 and IFNRα2 intracellular signaling domains heterodimerize in the presence of a ligand to promote growth and/or survival of a cell.

Illustrative examples of transmembrane domains suitable for use in particular multi-component cell surface receptor components contemplated herein include but are not limited to those isolated or obtained from IL2R, IL7R, IL12R, IL21R, CD4 or CD8a.

In particular embodiments, the system comprises a recombinant retrovirus comprising one or more transduction enhancers contemplated herein, optionally wherein the transduction enhancer is selected from the group consisting of a T cell activation receptor, a NK-cell activation receptor, and a costimulatory molecule.

In particular embodiments, the one or more transduction enhancers comprise one or more of CD80, CD86, CD137L, OX40L, and ICOSL.

In particular embodiments, a system comprises a recombinant retrovirus that transduces an immune effector cell, optionally a T cell, a natural killer (NK) cell, or a natural killer T (NKT) cell in vivo or ex vivo.

In particular embodiments, a first CAR comprises an antigen binding domain that specifically binds a cell-surface antigen selected from the group consisting of: alpha folate receptor (FRα), αvβ6 integrin, B cell maturation antigen (BCMA), B7-H3 (CD276), B7-H6, carbonic anhydrase IX (CAIX), CD16, CD 19, CD20, CD22, CD30, CD33, CD37, CD38, CD44, CD44v6, CD44v7/8, CD70, CD79a, CD79b, CD123, CD133, CD138, CD171, carcinoembryonic antigen (CEA), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant IP (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), G Protein-Coupled Receptor Class C Group 5 Member D (GPCR5D), EGFR family including ErbB2 (HER2), IL-10Rα, IL-13Rα2, Kappa, cancer/testis antigen 2 (LAGE-1A), Lambda, Lewis-Y (LeY), LI cell adhesion molecule (LI-CAM), melanoma antigen gene (MAGE)-A1, MAGE-A3, MAGE-A4, MAGE-A6, MAGE A10, melanoma antigen recognized by T cells 1 (MelanA or MARTI), Mesothelin (MSLN), MUC1, MUC16, MHC class I chain related proteins A (MICA), MHC class I chain related proteins B (MICB), neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), tumor endothelial marker 1 (TEM1/CD248), tumor endothelial marker 7-related (TEM7R), trophoblast glycoprotein (TPBG), UL 16-binding protein (ULBP) 1, ULBP2, ULBP3, ULBP4, ULBP5, ULBP6, vascular endothelial growth factor receptor 2 (VEGFR2), and Wilms tumor 1 (WT-1).

In particular embodiments, a second CAR comprises an antigen binding domain that specifically binds a cell-surface antigen selected from the group consisting of: FRα, αvβ6 integrin, BCMA, CD276, B7-H6, CAIX, CD 16, CD19, CD20, CD22, CD30, CD33, CD37, CD38, CD44, CD44v6, CD44v7/8, CD70, CD79a, CD79b, CD123, CD133, CD138, CD171, CEA, CLL-1, CS-1, CSPG4, CTAGE1, EGER, EGFRvIII, EGP2, EGP40, EPCAM, EPHA2, FAP, FCRL5, AchR, GD2, GD3, GPC3, GPCR5D, HER2, IL-10Rα, IL-13Rα2, LAGE-1A, Lambda, LeY, LI-CAM, MAGE-A1, MAGE-A3, MAGE-A4, MAGE-A6, MAGE A10, MelanA or MARTI, MSLN, MUC1, MUC16, MICA, MICB, NCAM, NY-ESO-1, PLAC1, PRAME, PSCA, PSMA, ROR1, SSX2, Survivin, TAG72, TEM1/CD248, TEM7R, TPBG, ULBP1, ULBP2, ULBP3, ULBP4, ULBP5, ULBP6, VEGFR2, and WT-1.

### D. Recombinant Retroviral Particles

Retroviruses include, without limitation, lentiviruses, gamma-retroviruses, and alpha-retroviruses. Retroviruses contemplated herein are used to deliver polynucleotides to immune effector cells. Lentiviruses are complex retroviruses, which, in addition to the common retroviral genes gag, pol, and env, contain other viral accessory genes. Illustrative retroviruses suitable for use in particular embodiments contemplated herein, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV) and lentivirus.

As used herein, the term “lentivirus” refers to a group (or genus) of complex retroviruses. Illustrative lentiviruses include, but are not limited to, HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV).

Recombinant retroviruses contemplated herein comprise genomes that lack one or more viral accessory genes, e.g., the genes env, vif, vpr, vpu and nef, thereby increasing the safety of the retroviruses. In particular embodiments, a recombinant virus contains two copies of a vector, a genomic RNA comprising backbone sequences derived from a lentivirus. It is understood that many different sources of retroviral and/or lentiviral sequences can be used, or combined and numerous substitutions and alterations in certain of the lentiviral sequences may be accommodated without impairing the ability of a vector to package polynucleotides into a recombinant retrovirus or retroviral particle and to transfer the polynucleotide into a target cell. Illustrative examples of recombinant retroviruses and retroviral vectors include, but are not limited to those described in Naldini et al., (1996a, 1996b, and 1998); Zufferey et al., (1997); Dull et al., 1998, U.S. Pat. Nos. 6,013,516; and 5,994,136, each of which are incorporated herein by reference in its entirety.

In particular embodiments, a lentivirus is manufactured using a third-generation lentiviral vector system comprising an envelope plasmid encoding a viral envelope glycoprotein (e.g., one or more mutated viral envelope glycoproteins that retain fusogenic activity and lack cognate receptor binding activity) and optionally, one or more non-viral membrane-bound tropism polypeptide and/or transduction enhancer polypeptides, two packaging plasmids (e.g., one encoding gag-pol and one encoding rev) and a transfer plasmid. The “transfer plasmid” encodes a viral genomic RNA (e.g., vector backbone) comprising the polynucleotide sequence that is delivered by recombinant lentivirus to a target cell. In particular embodiments, a transfer plasmid comprises one or more transgene sequences of interest flanked by long terminal repeat (LTR) sequences, which facilitate packaging, reverse transcription and integration of the viral vector and associated polynucleotide sequences into the host genome. Viral vectors contemplated herein are replication incompetent, i.e., lack the genetic elements necessary for generation of infective particles in the host cell. For example, the viral vector may be designed with a deletion of the 3′ LTR, rendering the virus “self-inactivating” (SIN).

In particular embodiments, the envelope plasmid and/or packaging plasmids further encode one or more non-viral membrane-bound tropism polypeptide and/or transduction enhancer polypeptides.

Recombinant retroviruses or retroviral particles are manufactured using a “packaging cell line.” In particular embodiments, recombinant retroviruses or retroviral particles are produced by transfecting a packaging cell line with the transfer plasmid, packaging plasmid(s), and envelope plasmid.

As used herein, the terms “retroviral vector” or “lentiviral vector” refer to a nucleic acid that encodes a retroviral or lentiviral genome comprising one or more polynucleotide sequences to be delivered to a target cell. In particular embodiments, a recombinant retrovirus or recombinant lentivirus comprises a genomic RNA (e.g., a vector), a lipid-bilayer envelope, and other accessory proteins including integrase, protease, and matrix protein. The vectors may further comprise one of more accessory elements to increase transduction efficiency (e.g., a FLAP element that includes the central polypurine tract and central termination sequences (cPPT and CTS)), viral packaging (e.g., a Psi (Y) packaging signal, a rev response element (RRE)), and/or other elements that increase therapeutic gene expression (e.g., poly (A) sequences), and may optionally comprise a woodchuck post-transcriptional regulatory element (WPRE) or hepatitis B virus post-transcriptional regulatory element (HPRE).

The efficiency of retroviral or lentiviral vector manufacturing may be assessed in various ways, including measurement of vector copy number (VCN) or vector genomes (vg) such as by quantitative polymerase chain reaction (qPCR), or titer of the virus in infectious units per milliliter (IU/mL). For example, the titer may be assessed using a functional assay performed on the cultured tumor cell line HT1080 or on HeLa cells.

In particular embodiments, a recombinant retrovirus contemplated herein comprises a vector encoding a promoter operably linked to one or more polynucleotides encoding one or more components of multi-component cell-surface receptor or macromolecular complex and a CAR. Illustrative promoters suitable for use in particular embodiments contemplated herein include, but are not limited to, an EF1α promoter, a cytomegalovirus (CMV) promoter, a CAG promoter, an SV40 promoter, an SV40/CD43 promoter, and a MND promoter.

In particular embodiments, a recombinant retrovirus comprises a viral envelope comprising or expressing one or more non-viral membrane-bound tropism polypeptides and/or one or more transduction enhancers. In particular embodiments, a recombinant retrovirus comprises one or more tagging proteins.

In particular embodiments, a recombinant retrovirus comprises a vector comprising a polynucleotide comprising, from 5′ to 3′: a 5′ long terminal repeat (LTR) comprising an R region and U5 region, a polynucleotide encoding a promoter, one or more polynucleotides encoding one or more components of multi-component cell-surface receptor or macromolecular complex and a CAR, a 3′ LTR comprising a U3 region and an R region, a polyadenylation signal and a poly(A) tail.

In particular embodiments, a recombinant retrovirus is a lentivirus comprising: a viral envelope that expresses a mutated viral envelope glycoprotein that retain fusogenic activity and lack cognate receptor binding activity, and a non-viral membrane-bound tropism polypeptide comprising an anti-CD3 scFv; and a SIN retroviral vector comprising an MNDU3 promoter, a transgene encoding one or more components of multi-component cell-surface receptor or macromolecular complex and an anti-CD19 CAR and/or anti-CD20 CAR, and/or an FRB polypeptide.

### E. Viral Envelope

In particular embodiments, a recombinant retrovirus comprises a viral envelope comprising one or more mutated viral envelope glycoproteins that mediate fusion of the viral particle and the target host cell but that do not bind its cognate receptor expressed on the target host cell and one or more non-viral membrane-bound tropism polypeptides.

In particular embodiments, one or more mutated viral envelope glycoproteins comprise a vesiculovirus envelope glycoprotein or one or more morbillivirus envelope glycoproteins or henipavirus envelope glycoproteins. In particular embodiments, the mutated viral envelope glycoprotein comprises a heterologous signal peptide.

In particular embodiments, one or more mutated viral envelope glycoproteins comprise a vesiculovirus envelope glycoprotein or one or more morbillivirus envelope glycoproteins or henipavirus envelope glycoproteins.

Illustrative examples of vesiculoviruses suitable for use in particular embodiments from which G glycoproteins can be isolated from include, but are not limited to vesicular stomatitis Alagoas virus (VSAV; Alagoas vesiculovirus), Carajás virus (CJSV; Carajas vesiculovirus), Chandipura virus (CHPV; Chandipura vesiculovirus), Cocal virus (COCV; Cocal vesiculovirus), vesicular stomatitis Indiana virus (VSIV, f.k.a. VSV; Indiana vesiculovirus), Isfahan virus (ISFV; Isfahan vesiculovirus), Maraba virus (MARAV; Maraba vesiculovirus), Morreton virus (MORV; Morreton vesiculovirus), vesicular stomatitis New Jersey virus (VSNJV; New Jersey vesiculovirus), and Piry virus (PIRYV; Piry vesiculovirus).

In particular embodiments, a vesiculovirus G envelope protein comprises one or more amino acid substitutions that that enable the polypeptide to mediate fusion of the viral particle and the target host cell but that ablate the polypeptide's ability to bind its cognate receptor expressed on the target host cell, e.g., LDL-R. In particular embodiments, a vesiculovirus G envelope protein comprises an amino acid sequence set forth in any one of SEQ ID NOs: 15-322 disclosed in U.S. patent application No. 20200216502, each said sequence incorporated by reference herein in its entirety, or an amino acid sequence at least 95%, 96%, 97%, 98%, or 99% identical thereto that enables the polypeptide to mediate fusion of the viral particle and the target host cell but that ablate the polypeptide's ability to bind its cognate receptor expressed on the target host cell, e.g., LDL-R. In particular embodiments, a vesiculovirus G envelope protein comprises amino acid substitution at positions 47 and/or 354 (or 358 in some strains, e.g., CJSV, VSNJV) with reference to the vesiculovirus G envelope protein lacking a signal peptide.

In particular embodiments, a vesiculovirus is vesicular stomatitis Indiana virus (VSIV). In particular embodiments, a mutated viral envelope glycoprotein is derived from a VSIV envelope glycoprotein (VSIV-G; e.g., SEQ ID NO: 1: KFTIVFPHNQKGNWKNVPSNYHYCPSSSDLNWHNDLIGTALQVKMPKSHKAIQA DGWMCHASKWVTTCDFRWYGPKYITHSIRSFTPSVEQCKESIEQTKQGTWLNPG FPPQSCGYATVTDAEAVIVQVTPHHVLVDEYTGEWVDSQFINGKCSNYICPTVHN STTWHSDYKVKGLCDSNLISMDITFFSEDGELSSLGKEGTGFRSNYFAYETGGKA CKMQYCKHWGVRLPSGVWFEMADKDLFAAARFPECPEGSSISAPSQTSVDVSLI QDVERILDYSLCQETWSKIRAGLPISPVDLSYLAPKNPGTGPAFTIINGTLKYFETR YIRVDIAAPILSRMVGMISGTTTERELWDDWAPYEDVEIGPNGVLRTSSGYKFPLY MIGHGMLDSDLHLSSKAQVFEHPHIQDAASQLPDDESLFFGDTGLSKNPIELVEG WFSSWKSSIASFFFIIGLIIGLFLVLRVGIHLCIKLKHTKKRQIYTDIEMNRLGK) or an amino acid sequence at least 95%, 96%, 97%, 98%, or 99% identical thereto comprising one or more modifications that enable the polypeptide to mediate fusion of the viral particle and a cell but that substantially ablate or ablate the polypeptide's ability to bind its cognate receptor expressed on a cell, e.g., LDL-R. In particular embodiments, a mutated viral envelope glycoprotein is derived from a VSIV-G polypeptide set forth in SEQ ID NO: 1 comprising L47I and/or H80Q amino acid substitutions, such substitutions being present in naturally occurring variants of VSIV.

In particular embodiments, a mutated VSIV-G envelope protein comprises one or more of: one or more amino acid substitutions at H8, N9, Q10, K47, K50, A51, S183, S179, N180, 1182, M184, Y209, 1347, T350, T352, E353, and R354 (substitution with any amino acid; a conservation substitution; a disruptive substitution; substitution with D, E, A, G, F, or Q; or substitution with A, G, F, or Q); an insertion of TT between N9 and Q10, an insertion of GGS between H8 and N9, an insertion of GGS between N9 and Q10, an insertion of TT between N208 and Y209, an insertion of GGS between P46 and K47, and an insertion of GGS between N208 and Y209; or a deletion of residues 1-8. In particular embodiments, a VSIV-G envelope protein comprises one or more amino acid substitutions at H8, K47, Y209, and R354. In particular embodiments, a VSIV-G envelope protein comprises one or more amino acid substitutions at H8, K47, Y209, and R354, wherein each substitution can be with A, G, F, or Q. Amino acid positions are with reference to a VSIV-G envelope protein that lacks a signal peptide, e.g., SEQ ID NO: 1. In particular embodiments, a mutated viral envelope glycoprotein is derived from a VSIV-G polypeptide set forth in SEQ ID NO: 1 comprising L47I and/or H80Q amino acid substitutions, such substitutions present in naturally occurring variants of VSIV.

In particular embodiments, a mutated VSIV-G polypeptide comprises one or more amino acid substitutions at K47, 1182, and/or R354 (substitution with any amino acid; a conservation substitution; a disruptive substitution; substitution with D, E, A, G, F, or Q; or substitution with A, G, F, or Q). In particular embodiments, a mutated VSIV-G polypeptide comprises amino acid substitutions at K47, I182, or R354; K47 and I182; K47 and R354; I182 and R354; or at K47, 1182, and R354 of SEQ ID NO: 1.

In particular embodiments, a mutated VSIV-G polypeptide comprises one or more of the following amino acid substitutions: K47A, K47Q, I182E, I182D, R354A, and/or

R354Q. In particular embodiments, a mutated VSIV-G polypeptide comprises the following amino acid substitutions: K47A, K47Q, I182E, I182D, R354A, or R354Q; K47A and I182E; K47A and I182D; K47Q and I182E; K47Q and I182D; I182E and R354A; I182E and R354Q; I182D and R354A; I182D and R354Q; K47A and R354A; K47A and R354Q; K47Q and R354A; K47Q and R354Q; K47A, I182E, and R354A; K47A, I182D, and R354A; K47Q, I182E, and R354A; K47Q, I182D, and R354A; K47A, I182E, and R354Q; K47A, I182D, and R354Q; K47Q, I182E, and R354Q; or K47Q, I182D, and R354Q of SEQ ID NO: 1.

In particular embodiments, a VSIV-G envelope protein comprises one or more amino acid substitutions at H8, K47, Y209, and R354. In particular embodiments, a VSIV-G envelope protein comprises one or more amino acid substitutions at H8, K47, Y209, and R354, wherein each substitution can be with A, G, F, or Q. In particular embodiments, a VSIV-G envelope protein comprises one or more amino acid substitutions at K47 and R354 (with reference to a VSIV-G envelope protein that lacks a signal peptide, e.g., SEQ ID NO: 1). In particular embodiments, a VSV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q. In preferred embodiments, a VSIV-G envelope comprises the amino acid substitutions K47Q or K47A and R354A or R354Q. Amino acid positions are with reference to a VSIV-G envelope protein that lacks a signal peptide.

In particular embodiments, a VSIV-G envelope protein comprises an amino acid sequence set forth in SEQ ID NO: 2 (wherein X₁=I, X₂=A, X₃=Q, and X₄=A; X₁=I, X₂=A, X₃=Q, and X₄=G; X₁=I, X₂=A, X₃=Q, and X₄=F; X₁=I, X₂=A, X₃=Q, and X₄=Q; X₁=L, X₂=A, X₃=Q, and X₄=A; X₁=L, X₂=A, X₃=Q, and X₄=G; X₁=L, X₂=A, X₃=Q, and X₄=F; X₁=L, X₂=A, X₃=Q, and X₄=Q; X₁=I, X₂=A, X₃=H, and X₄=A; X₁=I, X₂=A, X₃=H, and X₄=G; X₁=I, X₂=A, X₃=H, and X₄=F; X₁=I, X₂=A, X₃=H, and X₄=Q; X₁=L, X₂=A, X₃=H, and X₄=A; X₁=L, X₂=A, X₃=H, and X₄=G; X₁=L, X₂=A, X₃=H, and X₄=F; X₁=L, X₂=A, X₃=H, and X₄=Q; X₁=I, X₂=G, X₃=Q, and X₄=A; X₁=I, X₂=G, X₃=Q, and X₄=G; X₁=I, X₂=G, X₃=Q, and X₄=F; X₁=I, X₂=G, X₃=Q, and X₄=Q; X₁=L, X₂=G, X₃=Q, and X₄=A; X₁=L, X₂=G, X₃=Q, and X₄=G; X₁=L, X₂=G, X₃=Q, and X₄=F; X₁=L, X₂=G, X₃=Q, and X₄=Q; X₁=I, X₂=G, X₃=H, and X₄=A; X₁=I, X₂=G, X₃=H, and X₄=G; X₁=I, X₂=G, X₃=H, and X₄=F; X₁=I, X₂=G, X₃=H, and X₄=Q; X₁=L, X₂=G, X₃=H, and X₄=A; X₁=L, X₂=G, X₃=H, and X₄=G; X₁=L, X₂=G, X₃=H, and X₄=F; X₁=L, X₂=G, X₃=H, and X₄=Q; X₁=I, X₂=F, X₃=Q, and X₄=A; X₁=I, X₂=F, X₃=Q, and X₄=G; X₁=I, X₂=F, X₃=Q, and X₄=F; X₁=I, X₂=F, X₃=Q, and X₄=Q; X₁=L, X₂=F, X₃=Q, and X₄=A; X₁=L, X₂=F, X₃=Q, and X₄=G; X₁=L, X₂=F, X₃=Q, and X₄=F; X₁=L, X₂=F, X₃=Q, and X₄=Q; X₁=I, X₂=F, X₃=H, and X₄=A; X₁=I, X₂=F, X₃=H, and X₄=G; X₁=I, X₂=F, X₃=H, and X₄=F; X₁=I, X₂=F, X₃=H, and X₄=Q; X₁=L, X₂=F, X₃=H, and X₄=A; X₁=L, X₂=F, X₃=H, and X₄=G; X₁=L, X₂=F, X₃=H, and X₄=F; X₁=L, X₂=F, X₃=H, and X₄=Q; X₁=I, X₂=Q, X₃=Q, and X₄=A; X₁=I, X₂=Q, X₃=Q, and X₄=G; X₁=I, X₂=Q, X₃=Q, and X₄=F; X₁=I, X₂=Q, X₃=Q, and X₄=Q; X₁=L, X₂=Q, X₃=Q, and X₄=A; X₁=L, X₂=Q, X₃=Q, and X₄=G; X₁=L, X₂=Q, X₃=Q, and X₄=F; X₁=L, X₂=Q, X₃=Q, and X₄=Q; X₁=I, X₂=Q, X₃=H, and X₄=A; X₁=I, X₂=Q, X₃=H, and X₄=G; X₁=I, X₂=Q, X₃=H, and X₄=F; X₁=I, X₂=Q, X₃=H, and X₄=Q; X₁=L, X₂=Q, X₃=H, and X₄=A; X₁=L, X₂=Q, X₃=H, and X₄=G; X₁=L, X₂=Q, X₃=H, and X₄=F; and X₁=L, X₂=Q, X₃=H, and X₄=Q) or an amino acid sequence at least 95%, 96%, 97%, 98%, or 99% identical thereto that mediates fusion of the viral particle and the target host cell but that does not bind its cognate receptor expressed on the target host cell, e.g., LDL-R.

TABLE 1

| SEQ ID | |
|---|---|
| NO | Amino Acid Sequence |
| 2 | KFTIVFPHNQKGNWKNVPSNYHYCPSSSDLNWHNDLIGTAX₁ |
| | QVKMPX₂SHKAIQADGWMCHASKWVTTCDFRWYGPKYITX₃S |
| | IRSFTPSVEQCKESIEQTKQGTWLNPGFPPQSCGYATVTDA |
| | EAVIVQVTPHHVLVDEYTGEWVDSQFINGKCSNYICPTVHN |
| | STTWHSDYKVKGLCDSNLISMDITFFSEDGELSSLGKEGTG |
| | FRSNYFAYETGGKACKMQYCKHWGVRLPSGVWFEMADKDLF |
| | AAARFPECPEGSSISAPSQTSVDVSLIQDVERILDYSLCQE |
| | TWSKIRAGLPISPVDLSYLAPKNPGTGPAFTIINGTLKYFE |
| | TRYIRVDIAAPILSRMVGMISGTTTEX₄ELWDDWAPYEDVE |
| | IGPNGVLRTSSGYKFPLYMIGHGMLDSDLHLSSKAQVFEHP |
| | HIQDAASQLPDDESLFFGDTGLSKNPIELVEGWFSSWKSSI |
| | ASFFFIIGLIIGLFLVLRVGIHLCIKLKHTKKRQIYTDIEM |
| | NRLGK |

In particular embodiments, a mutated VSIV-G envelope protein comprises an amino acid sequence set forth in any one of SEQ ID NOs: 13-76 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R. In particular embodiments, a mutated VSIV-G envelope protein comprises an amino acid sequence set forth in any one of SEQ ID NOs: 61-76 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R. In particular embodiments, a mutated VSIV-G envelope protein comprises an amino acid sequence set forth in any one of SEQ ID NOs: 61, 65, 69, and 73 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R.

TABLE 2

| SEQ ID | |
|---|---|
| NO: | AMINO ACID SEQUENCE |
| 13 | KFTIVFPHNQKGNWKNVPSNYHYCPSSSDLNWHNDLIGTAIQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 14 | KFTIVFPHNQKGNWKNVPSNYHYCPSSSDLNWHNDLIGTAIQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 15 | KFTIVFPHNQKGNWKNVPSNYHYCPSSSDLNWHNDLIGTAIQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 16 | KFTIVFPHNQKGNWKNVPSNYHYCPSSSDLNWHNDLIGTAIQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 17 | KFTIVFPHNQKGNWKNVPSNYHYCPSSSDLNWHNDLIGTALQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 18 | KFTIVFPHNQKGNWKNVPSNYHYCPSSSDLNWHNDLIGTALQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 19 | KFTIVFPHNQKGNWKNVPSNYHYCPSSSDLNWHNDLIGTALQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 20 | KFTIVFPHNQKGNWKNVPSNYHYCPSSSDLNWHNDLIGTALQVKMPASHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 21 | KFTIVFPHNQKGNWKNVPSNYHYCPSSSDLNWHNDLIGTAIQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 22 | KFTIVFPHNQKGNWKNVPSNYHYCPSSSDLNWHNDLIGTAIQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 23 | KFTIVFPHNQKGNWKNVPSNYHYCPSSSDLNWHNDLIGTAIQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 24 | KFTIVFPHNQKGNWKNVPSNYHYCPSSSDLNWHNDLIGTAIQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 25 | KFTIVFPHNQKGNWKNVPSNYHYCPSSSDLNWHNDLIGTALQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 26 | KFTIVFPHNQKGNWKNVPSNYHYCPSSSDLNWHNDLIGTALQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 27 | KFTIVFPHNQKGNWKNVPSNYHYCPSSSDLNWHNDLIGTALQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 28 | KFTIVFPHNQKGNWKNVPSNYHYCPSSSDLNWHNDLIGTALQVKMPASHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 29 | KFTIVFPHNQKGNWKNVPSNYHYCPSSSDLNWHNDLIGTAIQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 30 | KFTIVFPHNQKGNWKNVPSNYHYCPSSSDLNWHNDLIGTAIQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 31 | KFTIVFPHNQKGNWKNVPSNYHYCPSSSDLNWHNDLIGTAIQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 32 | KFTIVFPHNQKGNWKNVPSNYHYCPSSSDLNWHNDLIGTAIQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 33 | KFTIVFPHNQKGNWKNVPSNYHYCPSSSDLNWHNDLIGTALQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 34 | KFTIVFPHNQKGNWKNVPSNYHYCPSSSDLNWHNDLIGTALQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 35 | KFTIVFPHNQKGNWKNVPSNYHYCPSSSDLNWHNDLIGTALQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 36 | KFTIVFPHNQKGNWKNVPSNYHYCPSSSDLNWHNDLIGTALQVKMPGSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 37 | KFTIVFPHNQKGNWKNVPSNYHYCPSSSDLNWHNDLIGTAIQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 38 | KFTIVFPHNQKGNWKNVPSNYHYCPSSSDLNWHNDLIGTAIQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 39 | KFTIVFPHNQKGNWKNVPSNYHYCPSSSDLNWHNDLIGTAIQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 40 | KFTIVFPHNQKGNWKNVPSNYHYCPSSSDLNWHNDLIGTAIQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 41 | KFTIVFPHNQKGNWKNVPSNYHYCPSSSDLNWHNDLIGTALQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 42 | KFTIVFPHNQKGNWKNVPSNYHYCPSSSDLNWHNDLIGTALQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 43 | KFTIVFPHNQKGNWKNVPSNYHYCPSSSDLNWHNDLIGTALQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 44 | KFTIVFPHNQKGNWKNVPSNYHYCPSSSDLNWHNDLIGTAIQVKMPGSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 45 | KFTIVFPHNQKGNWKNVPSNYHYCPSSSDLNWHNDLIGTAIQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 46 | KFTIVFPHNQKGNWKNVPSNYHYCPSSSDLNWHNDLIGTAIQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 47 | KFTIVFPHNQKGNWKNVPSNYHYCPSSSDLNWHNDLIGTAIQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 48 | KFTIVFPHNQKGNWKNVPSNYHYCPSSSDLNWHNDLIGTAIQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 49 | KFTIVFPHNQKGNWKNVPSNYHYCPSSSDLNWHNDLIGTALQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 50 | KFTIVFPHNQKGNWKNVPSNYHYCPSSSDLNWHNDLIGTALQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 51 | KFTIVFPHNQKGNWKNVPSNYHYCPSSSDLNWHNDLIGTALQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 52 | KFTIVFPHNQKGNWKNVPSNYHYCPSSSDLNWHNDLIGTALQVKMPFSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 53 | KFTIVFPHNQKGNWKNVPSNYHYCPSSSDLNWHNDLIGTAIQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 54 | KFTIVFPHNQKGNWKNVPSNYHYCPSSSDLNWHNDLIGTAIQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 55 | KFTIVFPHNQKGNWKNVPSNYHYCPSSSDLNWHNDLIGTAIQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 56 | KFTIVFPHNQKGNWKNVPSNYHYCPSSSDLNWHNDLIGTAIQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 57 | KFTIVFPHNQKGNWKNVPSNYHYCPSSSDLNWHNDLIGTALQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 58 | KFTIVFPHNQKGNWKNVPSNYHYCPSSSDLNWHNDLIGTALQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWESSWKSSIASFFFIIGLIIGLFLVLRVGIHLCIKLKHTKKRQIYTDIEMNRLGK |
| 59 | KFTIVFPHNQKGNWKNVPSNYHYCPSSSDLNWHNDLIGTALQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 60 | KFTIVFPHNQKGNWKNVPSNYHYCPSSSDLNWHNDLIGTALQVKMPFSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 61 | KFTIVFPHNQKGNWKNVPSNYHYCPSSSDLNWHNDLIGTAIQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 62 | KFTIVFPHNQKGNWKNVPSNYHYCPSSSDLNWHNDLIGTAIQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 63 | KFTIVFPHNQKGNWKNVPSNYHYCPSSSDLNWHNDLIGTAIQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 64 | KFTIVFPHNQKGNWKNVPSNYHYCPSSSDLNWHNDLIGTAIQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 65 | KFTIVFPHNQKGNWKNVPSNYHYCPSSSDLNWHNDLIGTALQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 66 | KFTIVFPHNQKGNWKNVPSNYHYCPSSSDLNWHNDLIGTALQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 67 | KFTIVFPHNQKGNWKNVPSNYHYCPSSSDLNWHNDLIGTALQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 68 | KFTIVFPHNQKGNWKNVPSNYHYCPSSSDLNWHNDLIGTALQVKMPQSHKAIQADGWMCHAS |
| | KWVTTCDFRWYGPKYITQSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 69 | KFTIVFPHNQKGNWKNVPSNYHYCPSSSDLNWHNDLIGTAIQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 70 | KFTIVFPHNQKGNWKNVPSNYHYCPSSSDLNWHNDLIGTAIQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 71 | KFTIVFPHNQKGNWKNVPSNYHYCPSSSDLNWHNDLIGTAIQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 72 | KFTIVFPHNQKGNWKNVPSNYHYCPSSSDLNWHNDLIGTAIQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 73 | KFTIVFPHNQKGNWKNVPSNYHYCPSSSDLNWHNDLIGTALQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEAELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 74 | KFTIVFPHNQKGNWKNVPSNYHYCPSSSDLNWHNDLIGTALQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEGELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 75 | KFTIVFPHNQKGNWKNVPSNYHYCPSSSDLNWHNDLIGTALQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEFELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |
| 76 | KFTIVFPHNQKGNWKNVPSNYHYCPSSSDLNWHNDLIGTALQVKMPQSHKAIQADGWMCHAS |
| | KWVTTCDFRWYGPKYITHSIRSFTPSVEQCKESIEQTKQGTWLNPGFPPQSCGYATVTDAEA |
| | VIVQVTPHHVLVDEYTGEWVDSQFINGKCSNYICPTVHNSTTWHSDYKVKGLCDSNLISMDI |
| | TFFSEDGELSSLGKEGTGFRSNYFAYETGGKACKMQYCKHWGVRLPSGVWFEMADKDLFAAA |
| | RFPECPEGSSISAPSQTSVDVSLIQDVERILDYSLCQETWSKIRAGLPISPVDLSYLAPKNP |
| | GTGPAFTIINGTLKYFETRYIRVDIAAPILSRMVGMISGTTTEQELWDDWAPYEDVEIGPNG |
| | VLRTSSGYKFPLYMIGHGMLDSDLHLSSKAQVFEHPHIQDAASQLPDDESLFFGDTGLSKNP |
| | IELVEGWFSSWKSSIASFFFIIGLIIGLFLVLRVGIHLCIKLKHTKKRQIYTDIEMNRLGK |

In particular embodiments, a vesiculovirus is cocal virus (COCV). In particular embodiments, a mutated viral envelope glycoprotein is derived from a COCV envelope glycoprotein (COCV-G; e.g., SEQ ID NO: 3: KFSIVFPQSQKGNWKNVPSSYHYCP SSSDQNWHNDLLGITMKVKMPKTHKAIQADGWMCHAAKWITTCDFRWYGPKY ITHSIHSIQPTSEQCKESIKQTKQGTWMSPGFPPQNCGYATVTDSVAVVVQATPHH VLVDEYTGEWIDSQFPNGKCETEECETVHNSTVWYSDYKVTGLCDATLVDTEITF FSEDGKKESIGKPNTGYRSNYFAYEKGDKVCKMNYCKHAGVRLPSGVWFEFVD QDVYAAAKLPECPVGATISAPTQTSVDVSLILDVERILDYSLCQETWSKIRSKQPV SPVDLSYLAPKNPGTGPAFTIINGTLKYFETRYIRIDIDNPIISKMVGKISGSQTEREL WTEWFPYEGVEIGPNGILKTPTGYKFPLFMIGHGMLDSDLHKTSQAEVFEHPHLA EAPKQLPEEETLFFGDTGISKNPVELIEGWFSSWKSTVVTFFFAIGVFILLYVVARI VIAVRYRYQGSNNKRIYNDIEMSRFRK) or an amino acid sequence at least 95%, 96%, 97%, 98%, or 99% identical thereto comprising one or more modifications that enable the polypeptide to mediate fusion of the viral particle and a cell but that substantially ablate or ablate the polypeptide's ability to bind its cognate receptor expressed on a cell, e.g., LDL-R. In particular embodiments, a COCV-G envelope protein comprises one or more amino acid substitutions at K47 and/or R354. In particular embodiments, a COCV-G envelope protein comprises one or more amino acid substitutions at K47 and/or R354, wherein each amino acid can be substituted with A, G, F, or Q. In particular embodiments, a COCV-G envelope protein comprises one or more amino acid substitutions at K47 and/or R354 (with reference to a COCV-G envelope protein that lacks a signal peptide, e.g., SEQ ID NO: 3). In particular embodiments, a COCV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q. In particular embodiments, a COCV-G envelope protein comprises an amino acid sequence set forth in SEQ ID NO: 4 (wherein X₁=A and X₂=A; X₁=A and X₂=G; X₁=A and X₂=F; X₁=A and X₂=Q; X₁=G and X₂=A; X₁=G and X₂=G; X₁=G and X₂=F; X₁=G and X₂=Q; X₁=F and X₂=A; X₁=F and X₂=G; X₁=F and X₂=F; X₁=F and X₂=Q; X₁=Q and X₂=A; X₁=Q and X₂=G; X₁=Q and X₂=F; or X₁=A and X₂=Q) or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R.

TABLE 3

| SEQ ID | |
|---|---|
| NO | Amino Acid Sequence |
| 4 | KFSIVFPQSQKGNWKNVPSSYHYCPSSSDQNWHNDLLGITMKVKMPX1THKAIQADGWMCHAAKW |
| | ITTCDFRWYGPKYITHSIHSIQPTSEQCKESIKQTKQGTWMSPGFPPQNCGYATVTDSVAVVVQA |
| | TPHHVLVDEYTGEWIDSQFPNGKCETEECETVHNSTVWYSDYKVTGLCDATLVDTEITFFSEDGK |
| | KESIGKPNTGYRSNYFAYEKGDKVCKMNYCKHAGVRLPSGVWFEFVDQDVYAAAKLPECPVGATI |
| | SAPTQTSVDVSLILDVERILDYSLCQETWSKIRSKQPVSPVDLSYLAPKNPGTGPAFTIINGTLK |
| | YFETRYIRIDIDNPIISKMVGKISGSQTEX2ELWTEWFPYEGVEIGPNGILKTPTGYKFPLEMIG |
| | HGMLDSDLHKTSQAEVFEHPHLAEAPKQLPEEETLFFGDTGISKNPVELIEGWESSWKSTVVTFF |
| | FAIGVFILLYVVARIVIAVRYRYQGSNNKRIYNDIEMSRFRK |

In particular embodiments, a mutated COCV-G envelope protein comprises an amino acid sequence set forth in any one of SEQ ID NOs: 77-92 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R. In particular embodiments, a mutated COCV-G envelope protein comprises an amino acid sequence set forth in any one of SEQ ID NOs: 89-92 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R. In particular embodiments, a mutated VSIV-G envelope protein comprises an amino acid sequence set forth in SEQ ID NOs: 89 or 92 or an amino acid sequence at least 95%, 96%, 97%, 98%, or 99% identical thereto that mediates fusion of the viral particle and a cell but that substantially ablates or ablates the polypeptide's ability to bind its cognate receptor expressed on a cell, e.g., LDL-R.

TABLE 4

| SEQ ID | |
|---|---|
| NO: | AMINO ACID SEQUENCE |
| 77 | KFSIVFPQSQKGNWKNVPSSYHYCPSSSDQNWHNDLLGITMKVKMPATHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEAELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 78 | KFSIVFPQSQKGNWKNVPSSYHYCPSSSDQNWHNDLLGITMKVKMPATHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEGELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 79 | KFSIVFPQSQKGNWKNVPSSYHYCPSSSDQNWHNDLLGITMKVKMPATHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEFELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 80 | KFSIVFPQSQKGNWKNVPSSYHYCPSSSDQNWHNDLLGITMKVKMPATHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEQELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 81 | KFSIVFPQSQKGNWKNVPSSYHYCPSSSDQNWHNDLLGITMKVKMPGTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEAELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 82 | KFSIVFPQSQKGNWKNVPSSYHYCPSSSDQNWHNDLLGITMKVKMPGTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEGELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 83 | KFSIVFPQSQKGNWKNVPSSYHYCPSSSDQNWHNDLLGITMKVKMPGTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEFELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 84 | KFSIVFPQSQKGNWKNVPSSYHYCPSSSDQNWHNDLLGITMKVKMPGTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEQELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 85 | KFSIVFPQSQKGNWKNVPSSYHYCPSSSDQNWHNDLLGITMKVKMPFTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEAELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 86 | KFSIVFPQSQKGNWKNVPSSYHYCPSSSDQNWHNDLLGITMKVKMPFTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEGELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 87 | KFSIVFPQSQKGNWKNVPSSYHYCPSSSDQNWHNDLLGITMKVKMPFTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEFELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 88 | KFSIVFPQSQKGNWKNVPSSYHYCPSSSDQNWHNDLLGITMKVKMPFTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEQELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 89 | KFSIVFPQSQKGNWKNVPSSYHYCPSSSDQNWHNDLLGITMKVKMPQTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEAELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 90 | KFSIVFPQSQKGNWKNVPSSYHYCPSSSDQNWHNDLLGITMKVKMPQTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEGELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 91 | KFSIVFPQSQKGNWKNVPSSYHYCPSSSDQNWHNDLLGITMKVKMPQTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEFELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |
| 92 | KFSIVFPQSQKGNWKNVPSSYHYCPSSSDQNWHNDLLGITMKVKMPQTHKAIQADGWMCHAA |
| | KWITTCDFRWYGPKYITHSIHSIQPTSEQCKESIKQTKQGTWMSPGFPPQNCGYATVTDSVA |
| | VVVQATPHHVLVDEYTGEWIDSQFPNGKCETEECETVHNSTVWYSDYKVTGLCDATLVDTEI |
| | TFFSEDGKKESIGKPNTGYRSNYFAYEKGDKVCKMNYCKHAGVRLPSGVWFEFVDQDVYAAA |
| | KLPECPVGATISAPTQTSVDVSLILDVERILDYSLCQETWSKIRSKQPVSPVDLSYLAPKNP |
| | GTGPAFTIINGTLKYFETRYIRIDIDNPIISKMVGKISGSQTEQELWTEWFPYEGVEIGPNG |
| | ILKTPTGYKFPLFMIGHGMLDSDLHKTSQAEVFEHPHLAEAPKQLPEEETLFFGDTGISKNP |
| | VELIEGWFSSWKSTVVTFFFAIGVFILLYVVARIVIAVRYRYQGSNNKRIYNDIEMSRFRK |

In particular embodiments, one or more mutated morbillivirus envelope glycoproteins are derived from measles virus F (MV-F) and measles virus H (MV-H). In particular embodiments, a recombinant particle comprises one or more measles virus viral envelope glycoproteins modified to lack cell binding activity and retain fusogenic activity. In some embodiments, a recombinant particle comprises a modified MV-F glycoprotein and an MV-H glycoprotein modified to lack cell binding activity and retain fusogenic activity.

In particular embodiments, one or more mutated morbillivirus envelope glycoproteins are derived from measles virus F (MV-F) polypeptide (e.g., SEQ ID NO: 5: QIHWGNLSKIGVVGIGSASYKVMTRSSHQSLVIKLMPNITLLNNCTRVEIAEYRRL LRTVLEPIRDALNAMTQNIRPVQSVASSRRHKRFAGVVLAGAALGVATAAQITA GIALHQSMLNSQAIDNLRASLETTNQAIEAIRQAGQEMILAVQGVODYINNELIPS MNQLSCDLIGQKLGLKLLRYYTEILSLFGPSLRDPISAEISIQALSYALGGDINKVLE KLGYSGGDLLGILESRGIKARITHVDTESYFIVLSIAYPTLSEIKGVIVHRLEGVSYN IGSQEWYTTVPKYVATQGYLISNFDESSCTFMPEGTVCSQNALYPMSPLLQECLR GSTKSCARTLVSGSFGNRFILSQGNLIANCASILCKCYTTGTIINQDPDKILTYIAAD HCPVVEVNGVTIQVGSRRYPDAVYLHRIDLGPPISLERLDVGTNLGNAIAKLEDA KELLESSDQILRSMKGLSSTSIVYILIAVCLGGLIGIPALICCCRGR) or an amino acid sequence at least 95%, 96%, 97%, 98%, or 99% identical thereto and a measles virus H (MV-H) polypeptide (e.g., SEQ ID NO: 6: MGSRIVINREHLMIDRPYVLLA VLFVMFLSLIGLLAIAGIRLHRAAIYTAEIHKSLST NLDVTNSIEHQVKDVLTPLFKIIGDEVGLRTPQRFTDLVKFISDKIKFLNPDREYDF RDLTWCINPPERIKLDYDQYCADVAAEELMNALVNSTLLETRTTNQFLAVSKGN CSGPTTIRGQFSNMSLSLLDLYLGRGYNVSSIVTMTSQGMYGGTYLVEKPNLSSK RSELSQLSMYRVFEVGVIRNPGLGAPVFHMTNYLEQPVSNDLSNCMVALGELKL AALCHGEDSITIPYQGSGKGVSFQLVKLGVWKSPTDMQSWVPLSTDDPVIDRLYL SSHRGVIADNQAKWAVPTTRTDDKLRMETC.FQQACKGKIQALCENPEWAPLKD NRIPSYGVLSVDLSLTVELKIKIASGFGPLITHGSGMDLYKSNHNNVYWLTIPPMK NLALGVINTLEWIPRFKVSPYLFTVPIKEAGGDCHAPTYLPAEVDGDVKLSSNLVI LPGQDLQYVLATYDTSRVEHAVVYYVYSPSRSFSYFYPFRLPIKGVPIELQVECFT WDQKLWCRHFCVLADSESGGHITHSGMVGMGVSCTVTREDGTN) or an amino acid sequence at least 95%, 96%, 97%, 98%, or 99% identical thereto, wherein the MV-H protein lacks cell binding activity and retains fusogenic activity. In particular embodiments, the MV-H polypeptide comprises one or more amino acid substitutions at positions Y463, R515, S530, and F531 of a MV-H polypeptide (e.g., SEQ ID NO 6). In particular embodiments, the MV-H polypeptide comprises one or more of the amino acid substitutions Y463A, R515A, S530L, and F531S in an MV-H polypeptide (e.g., SEQ ID NO: 7: MGSRIVINREHLMIDRPYVLLA VLFVMFLSLIGLLAIAGIRLHRAAIYTAEIHKSLST NLDVTNSIEHQVKDVLTPLFKIIGDEVGLRTPQRFTDLVKFISDKIKFLNPDREYDF RDLTWCINPPERIKLDYDQYCADVAAEELMNALVNSTLLETRTTNQFLAVSKGN CSGPTTIRGQFSNMSLSLLDLYLGRGYNVSSIVTMTSQGMYGGTYLVEKPNLSSK RSELSQLSMYRVFEVGVIRNPGLGAPVFHMTNYLEQPVSNDLSNCMVALGELKL AALCHGEDSITIPYQGSGKGVSFQLVKLGVWKSPTDMQSWVPLSTDDPVIDRLYL SSHRGVIADNQAKWAVPTTRTDDKLRMETC.FQQACKGKIQALCENPEWAPLKD NRIPSYGVLSVDLSLTVELKIKIASGFGPLITHGSGMDLYKSNHNNVYWLTIPPMK NLALGVINTLEWIPRFKVSPALFNVPIKEAGGDCHAPTYLPAEVDGDVKLSSNLVI LPGQDLQYVLATYDTSAVEHAVVYYVYSPSRLSSYFYPFRLPIKGVPIELQVECFT WDQKLWCRHFCVLADSESGGHITHSGMVGMGVSCTVTREDGTN) or an amino acid sequence at least 95%, 96%, 97%, 98%, or 99% identical thereto.

In particular embodiments, one or more mutated henipaviruses envelope glycoproteins are derived from nipah virus F (NiV-F) and nipah virus G (NiV-G). In particular embodiments, a recombinant particle comprises one or more nipah virus viral envelope glycoproteins modified to lack cell binding activity and retain fusogenic activity. In some embodiments, a recombinant particle comprises a modified NiV-F glycoprotein and a NiV-G glycoprotein modified to lack cell binding activity and retain fusogenic activity.

In particular embodiments, one or more mutated henipavirus envelope glycoproteins are derived from a nipah virus F (NiV-F) polypeptide (e.g., SEQ ID NO: 8: LHYEKLSKIGLVKGVTRKYKIKSNPLTKDIVIKMIPNVSNMSQCTGSVMENYKTR LNGILTPIKGALEIYKNNTHDLVGDVRLAGVIMAGVAIGIATAAQITAGVALYEA MKNADNINKLKSSIESTNEAVVKLQETAEKTVYVLTALQDYINTNLVPTIDKISCK QTELSLDLALSKYLSDLLFVFGPNLQDPVSNSMTIQAISQAFGGNYETLLRTLGYA TEDFDDLLESDSITGQIIYVDLSSYYIIVRVYFPILTEIQQAYIQELLPVSFNNDNSEW ISIVPNFILVRNTLISNIEIGFCLITKRSVICNQDYATPMTNNMRECLTGSTEKCPREL VVSSHVPRFALSNGVLFANCISVTCQCQTTGRAISQSGEQTLLMIDNTTCPTAVLG NVIISLGKYLGSVNYNSEGIAIGPPVFTDKVDISSQISSMNQSLQQSKDYIKEAQRL LDTVNPSLISMLSMIILYVLSIASLCIGLITFISFIIVEKKRNT) or an amino acid sequence at least 95%, 96%, 97%, 98%, or 99% identical thereto and a nipah virus G (NiV-G) polypeptide (e.g., SEQ ID NO: 9: MKKINEGLLDSKILSAFNTVIALLGSIVIIVMNIMIIQNYTRSTDNQAVIKDALQGIQ QQIKGLADKIGTEIGPKVSLIDTSSTITIPANIGLLGSKISQSTASINENVNEKCKFTL PPLKIHECNISCPNPLPFREYRPQTEGVSNLVGLPNNICLQKTSNQILKPKLISYTLP VVGQSGTCITDPLLAMDEGYFAYSHLERIGSCSRGVSKQRIIGVGEVLDRGDEVPS LFMTNVWTPPNPNTVYHCSAVYNNEFYYVLCAVSTVGDPILNSTYWSGSLMMT RLAVKPKSNGGGYNQHQLALRSIEKGRYDKVMPYGPSGIKQGDTLYFPAVGFLV RTEFKYNDSNCPITKCQYSKPENCRLSMGIRPNSHYILRSGLLKYNLSDGENPKVV FIEISDQRLSIGSPSKIYDSLGQPVFYQASFSWDTMIKFGDVLTVNPLVVNWRNNT VISRPGQSQCPRFNTCPEICWEGVYNDAFLIDRINWISAGVFLDSNQTAENPVFTV FKDNEILYRAQLASEDTNAQKTITNCFLLKNKIWCISLVEIYDTGDNVIRPKLFAV KIPEQCT) or an amino acid sequence at least 95%, 96%, 97%, 98%, or 99% identical thereto, wherein the NiV-G polypeptide lacks cell binding activity and retains fusogenic activity. In particular embodiments, the NiV-G polypeptide comprises one or more amino acid substitutions at positions E468, W471, Q497, and E500 of an NiV-G polypeptide (e.g., SEQ ID NO: 9). In particular embodiments, the NiV-G polypeptide comprises one or more of the amino acid substitutions E468A, W471A, Q497A, and E500A in an NiV-G polypeptide (e.g., SEQ ID NO: 10: MKKINEGLLDSKILSAFNTVIALLGSIVIIVMNIMIIQNYTRSTDNQAVIKDALQGIQ QQIKGLADKIGTEIGPKVSLIDTSSTITIPANIGLLGSKISQSTASINENVNEKCKFTL PPLKIHECNISCPNPLPFREYRPQTEGVSNLVGLPNNICLQKTSNQILKPKLISYTLP VVGQSGTCITDPLLAMDEGYFAYSHLERIGSCSRGVSKQRIIGVGEVLDRGDEVPS LFMTNVWTPPNPNTVYHCSAVYNNEFYYVLCAVSTVGDPILNSTYWSGSLMMT RLAVKPKSNGGGYNQHQLALRSIEKGRYDKVMPYGPSGIKQGDTLYFPAVGFLV RTEFKYNDSNCPITKCQYSKPENCRLSMGIRPNSHYILRSGLLKYNLSDGENPKVV FIEISDQRLSIGSPSKIYDSLGQPVFYQASFSWDTMIKFGDVLTVNPLVVNWRNNT VISRPGQSQCPRFNTCPAICAEGVYNDAFLIDRINWISAGVFLDSNATAANPVFTV FKDNEILYRAQLASEDTNAQKTITNCFLLKNKIWCISLVEIYDTGDNVIRPKLFAV KIPEQCT) or an amino acid sequence at least 95%, 96%, 97%, 98%, or 99% identical thereto, wherein the NiV-G polypeptide lacks cell binding activity and retains fusogenic activity.

In particular embodiments, a recombinant retrovirus contemplated herein comprises an outer surface, a lipid bilayer, cell membrane, or viral envelope comprising or expressing one or more non-viral membrane bound tropism polypeptides. A “tropism polypeptide” is a polypeptide that binds one or more antigens on a target host cell. A “non-viral membrane bound tropism polypeptide” is a polypeptide that binds one or more antigens on a target host cell; that is not native to, or derived from, either in whole or in part, a virus; and that is attached to a lipid bilayer, cell membrane, or viral envelope.

In particular embodiments, a non-viral membrane bound tropism polypeptide comprises an extracellular antigen targeting domain, a spacer polypeptide, and a transmembrane domain. In particular embodiments, an extracellular antigen targeting domain binds an antigen expressed on an immune effector cell. In particular embodiments, an extracellular antigen targeting domain comprises an antibody or antigen binding fragment thereof that binds an antigen expressed on the surface of an immune effector cell. In particular embodiments, an extracellular antigen targeting domain binds an antigen expressed on an immune effector cell.

In particular embodiments, the extracellular antigen targeting domain binds CD3δ, CD3ε CD3γ, CD4, CD5, CD7, CD8α, or CD8β. In particular embodiments, the extracellular antigen targeting domain comprises an scFv, a murine scFv, a humanized scFv, or a human scFv that binds CD3δ, CD3ε CD3γ, CD4, CD5, CD7, CD8α, or CD8β.

In particular embodiments, a tropism polypeptide comprises an anti-CD3ε antibody or antigen binding fragment thereof. Illustrative examples of anti-CD3ε antibodies or antigen binding fragments thereof suitable for using in particular embodiments include scFvs or other antigen binding fragments isolated from OKT3, UCHT1, YTH12.5, and TR66, and variants thereof, e.g., teplizumab and variants having at least 75%, at least 80%, at least 85%, at least 90%, at least 95%, or at least 99% identity thereto.

Illustrative anti-CD3 scFvs include the following amino acid sequences.

| SEQ ID | |
|---|---|
| NO: | Amino Acid Sequence |
| 11 | DIQMTQSPSSLSASVGDRVTITCSASSSVSYMNWYQQTPGKAP |
| | KRWIYDTSKLASGVPSRFSGSGSGTDYTFTISSLQPEDIATYY |
| | CQQWSSNPFTFGQGTKLQITRTSGGGGSGGGGSGGGGSQVQLV |
| | QSGGGVVQPGRSLRLSCKASGYTFTRYTMHWVRQAPGKGLEWI |
| | GYINPSRGYTNYNQKVKDRFTISRDNSKNTAFLQMDSLRPEDT |
| | GVYFCARYYDDHYCLDYWGQGTPVTVSS |
| 12 | DIVMTQSPAIMSASPGEKVTMTCSASSSVSYMNWYQQKSGTSP |
| | KRWIYDTSKLASGVPAHFRGSGSGTSYSLTISGMEAEDAATYY |
| | CQQWSSNPFTFGSGTKLEIKGGGGGGGGSGGGGSQVQLLESGA |
| | ELARPGASVKMSCKASGYTFTRYTMHWVKQRPGQGLEWIGYIN |
| | PSRGYTNYNQKFKDKATLTTDKSSSTAYMQLSSLTSEDSAVYY |
| | CAGYYDDHYCLDYWGQGTLVTVSS |

In particular embodiments, a tropism polypeptide comprises a Gaussia luciferase signal peptide, an anti-CD3 scFv, a hinge domain (e.g., isolated from an IgG1 Fc region, an IgG1hinge or a human CD8 stalk, the mouse CD8a stalk, or Glycophorin A) or linker (e.g., a (G4S)₁₋₅), a transmembrane domain (e.g., isolated from CD3, CD4, CD8α, CD28, or Glycophorin A), and a cytoplasmic tail (e.g., isolated from Glycophorin A). Illustrative examples of polypeptides suitable for use in particular embodiments are disclosed in WO202107678 and WO2022164935, each of which is incorporated herein by reference in its entirety.

### F. Transduction Enhancers

In some embodiments, recombinant retroviral particles contemplated herein comprise a viral envelope comprising or expressing one or more transduction enhancers. A “transduction enhancer” as used herein refers to a non-viral membrane-bound protein that activates T cells. In particular embodiments, a transduction enhancer comprises a mitogenic and/or cytokine-based domain. In particular embodiments, a transduction enhancer comprises T cell activation ligands or receptors, NK cell activation receptors, co-stimulatory molecules, or portions thereof.

### 1. Mitogenic Transduction Enhancers

In particular embodiments, a recombinant retrovirus contemplated herein comprises a viral envelope comprising or expressing a mitogenic transduction enhancer. In some embodiments, the mitogenic transduction enhancer is derived from the host cell during recombinant retrovirus production. In some embodiments, a mitogenic transduction enhancer is made by the packaging cell and expressed at the cell surface. When the nascent retroviral vector particle buds from the host cell membrane, the mitogenic transduction enhancer is incorporated in the viral envelope as part of the packaging cell-derived lipid bilayer.

In some embodiments, a transduction enhancer is host cell derived. The term “host cell derived” means that the mitogenic transduction enhancer is derived from the host cell. In some embodiments, a mitogenic transduction enhancer may have the structure: M-S-TM-CT, in which M is a mitogenic domain; S is an optional spacer domain TM is a transmembrane domain, and CT is an optional cytoplasmic tail.

### 2. Transduction Enhancer Mitogenic Domains

A mitogenic domain is the part of the mitogenic transduction enhancer which causes T cell activation. It may bind or otherwise interact, directly or indirectly, with a T cell, leading to T cell activation. In particular embodiments, the mitogenic domain binds a T cell surface antigen including but not limited to the alpha or beta chains of a TCR, CD28, CD134 (OX40), CD137 (4-1BB), and CD278 (ICOS).

In particular embodiments, the mitogenic domain comprises an antibody or antigen binding fragment thereof that specifically binds a T cell surface antigen. In particular embodiments, the mitogenic domain comprises an antibody or antigen binding fragment thereof that specifically binds the alpha or beta chains of a TCR, CD28, CD134 (OX40), CD137 (4-1BB), and CD278 (ICOS).

Illustrative examples of anti-CD28 antibodies or antigen binding fragments thereof suitable for use in particular embodiments include those derived from 9.3, 15E8, CD28.2, 10F3, and TGN1412 and humanized variants thereof.

In particular embodiments, a mitogenic domain comprises all or part of a co-stimulatory molecule including but not limited to CD80, CD86, OX40L, 4-1BBL, and ICOSL.

### 3. Transduction Enhancer Spacer Domains

In particular embodiments, a mitogenic transduction enhancer and/or cytokine-based transduction enhancer comprises a spacer domain that connects the mitogenic domain with the transmembrane domain. A flexible spacer allows the antigen binding domain to orient in different directions to facilitate binding. In particular embodiments, the spacer domain comprises an IgG1 Fc region, an IgG1 hinge, a human CD8a stalk or the mouse CD8a stalk, a CD2 ectodomain, or a CD34 ectodomain. In particular embodiments, the spacer domain comprises an alternative linker sequence which has similar length and/or domain spacing properties as an IgG1 Fc region, an IgG1 hinge or a CD8a stalk. In particular embodiments, a human IgG1 spacer may be altered to remove Fc binding motifs. In some embodiments, the spacer domain may be derived from a human protein.

### 4. Transduction Enhancer Transmembrane Domains

In particular embodiments, the transduction enhancer comprises a transmembrane domain that tethers the transduction enhancer and/or cytokine-based transduction enhancer to the membrane. In particular embodiments, the transmembrane domain comprises a hydrophobic alpha helix. In particular embodiments, the transmembrane domain may be derived from CD3, CD4, CD8a, or CD28. In some embodiments, the transmembrane domain is derived from a human protein.

In some embodiments, a transduction enhancer does not comprise a transmembrane domain but instead comprises a membrane-targeting domain such as a GPI anchor. GPI anchoring is a post-translational modification which occurs in the endoplasmic reticulum. Preassembled GPI anchor precursors are transferred to proteins bearing a C-terminal GPI signal sequence. During processing, the GPI anchor replaces the GPI signal sequence and is linked to the target protein via an amide bond. The GPI anchor targets the mature protein to the membrane. In some embodiments, the present tagging protein comprises a GPI signal sequence.

### 5. Cytokine-Based Transduction Enhancers

In particular embodiments, a recombinant retrovirus contemplated herein comprises a viral envelope comprising or expressing a cytokine-based transduction enhancer. In some embodiments, the cytokine-based transduction enhancer is derived from the host cell during viral vector production. In some embodiments, the cytokine-based transduction enhancer is made by the host cell and expressed at the cell surface. When the nascent retroviral vector particle buds from the host cell membrane, the cytokine-based transduction enhancer may be incorporated in the viral envelope as part of the packaging cell-derived lipid bilayer.

In particular embodiments, the cytokine-based transduction enhancer comprises a cytokine domain and a transmembrane domain. It may have the structure C-S-TM, where C is the cytokine domain, S is an optional spacer domain and TM is the transmembrane domain. The spacer domain and transmembrane domains are as defined above.

### 6. Transduction Enhancer Cytokine Domains

In particular embodiments, a cytokine domain comprises part, or all, of a T cell activating cytokine. Illustrative examples of cytokine domains suitable for use in particular embodiments contemplated herein include but are not limited to those isolated or obtained from IL2, IL7, and IL15. In particular embodiments, a cytokine domain comprises a fragment of a cytokine that binds to its cognate receptor and activates T cells.

### 7. Viral Vectors Comprising Transduction Enhancers

In particular embodiments, a recombinant retrovirus or lentivirus comprises a viral envelope comprising or expression one or more mutated viral envelope glycoproteins that retain fusogenic activity and lack cognate receptor binding activity, a non-viral membrane-bound tropism polypeptide, a mitogenic transduction enhancer which comprises a mitogenic domain and a transmembrane domain; and/or a cytokine-based transduction enhancer which comprises a cytokine domain and a transmembrane domain.

In particular embodiments, a recombinant retrovirus or lentivirus comprises a viral envelope comprising or expression one or more mutated viral envelope glycoproteins that retain fusogenic activity and lack cognate receptor binding activity; a non-viral membrane-bound tropism polypeptide; a mitogenic transduction enhancer having the structure: M-S-TM, in which M is a mitogenic domain; S is an optional spacer domain and TM is a transmembrane domain; and/or a cytokine-based transduction enhancer comprising a cytokine domain and a transmembrane domain.

In particular embodiments, the mitogenic transduction enhancer binds an activating T cell surface antigen, e.g., the alpha or beta chains of a TCR, CD28, CD134, CD137, CD278. In particular embodiments, the mitogenic transduction enhancer comprises an agonist for such an activating T cell surface antigen.

In particular embodiments, the mitogenic transduction enhancer comprises the binding domain from an antibody such as 9.3, 15E8, TGN1412; or a costimulatory molecule such as OX40L, 4-1BBL, or ICOSL.

In particular embodiments, a recombinant retrovirus or lentivirus comprises a viral envelope comprising or expressing one or more mutated viral envelope glycoproteins that retain fusogenic activity and lack cognate receptor binding activity, a non-viral membrane-bound tropism polypeptide, and two or more mitogenic transduction enhancers. In one embodiment, a recombinant retrovirus or lentivirus comprises a viral envelope comprising or expressing one or more mutated viral envelope glycoproteins that retain fusogenic activity and lack cognate receptor binding activity, a non-viral membrane-bound tropism polypeptide that binds CD3 (e.g., CD3ε) and a mitogenic transduction enhancer that binds CD28.

In particular embodiments, a recombinant retrovirus or lentivirus comprises a viral envelope comprising or expressing one or more mutated viral envelope glycoproteins that retain fusogenic activity and lack cognate receptor binding activity, a non-viral membrane-bound tropism polypeptide that binds CD3; and a mitogenic transduction enhancer that binds CD28.

In particular embodiments, a recombinant retrovirus or lentivirus comprises a viral envelope comprising or expressing one or more mutated viral envelope glycoproteins that retain fusogenic activity and lack cognate receptor binding activity, a non-viral membrane-bound tropism polypeptide that binds CD3; a mitogenic transduction enhancer that binds CD28; and a cytokine-based transduction enhancer comprising IL2.

In particular embodiments, a recombinant retrovirus or lentivirus comprises a viral envelope comprising or expressing one or more mutated viral envelope glycoproteins that retain fusogenic activity and lack cognate receptor binding activity, a non-viral membrane-bound tropism polypeptide that binds CD3; a mitogenic transduction enhancer that binds CD28; and a cytokine-based transduction enhancer comprising IL7; and/or a cytokine-based transduction enhancer comprising IL15.

### G. Chimeric Antigen Receptor

In particular embodiments, a system comprises one or more retroviral vectors comprising one or more polynucleotides encoding a CAR. A “chimeric antigen receptor” or “CAR” refers to a synthetically designed receptor comprising an antigen binding domain of an antibody or other protein sequence that binds to a molecule expressed or displayed on a target cell, a transmembrane domain, one or more intracellular signaling domains, e.g., one or more co-stimulatory domains and/or a primary signaling domain. The antigen binding domain is linked via a hinge domain to a transmembrane domain which is in turn linked, optionally by a spacer or linker domain, to one or more T cell intracellular signaling domains. CARs are engineered antigen receptors that can enable an immune receptor cell specifically recognize a target cell. In particular embodiments, the hinge or spacer/linker domain(s) are selected (e.g., for a particular length of amino acids) to achieve desired binding and/or functional characteristics of the CAR.

a. Antigen Binding Domain

In particular embodiments, the antigen binding fragment comprises a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv) 2, a minibody, a diabody, a triabody, a tetrabody, a disulfide stabilized Fv protein (“dsFv”), a single-domain antibody (sdAb, a camelid VHH, Nanobody) or centyrin.

In particular embodiments, the antigen binding domain comprises an scFv or one or more VHHs.

In particular embodiments, the antigen binding domain binds an antigen selected from the group consisting of: FRα, αvβ6 integrin, BCMA, CD276, B7-H6, CAIX, CD 16, CD19, CD20, CD22, CD30, CD33, CD37, CD38, CD44, CD44v6, CD44v7/8, CD70, CD79a, CD79b, CD123, CD133, CD138, CD171, CEA, CLL-1, CS-1, CSPG4, CTAGE1, EGER, EGFRVIII, EGP2, EGP40, EPCAM, EPHA2, FAP, FCRL5, AchR, GD2, GD3, GPC3, GPCR5D, HER2, IL-10Rα, IL-13Rα2, LAGE-1A, Lambda, LeY, L1-CAM, MAGE-A1, MAGE-A3, MAGE-A4, MAGE-A6, MAGE A10, MelanA or MARTI, MSLN, MUC1, MUC16, MICA, MICB, NCAM, NY-ESO-1, PLAC1, PRAME, PSCA, PSMA, ROR1, SSX2, Survivin, TAG72, TEM1/CD248, TEM7R, TPBG, ULBP1, ULBP2, ULBP3, ULBP4, ULBP5, ULBP6, VEGFR2, and WT-1.

In particular embodiments, the antigen binding domain binds CD19.

In particular embodiments, the antigen binding domain binds CD20.

In particular embodiments, the antigen binding domain binds BCMA.

In particular embodiments, the antigen is expressed on a malignant B cell. In particular embodiments, the B cell malignancy is diffuse large B-cell lymphoma (DLBCL) cell, Burkitt's type large B-cell lymphoma (B-LBL) cell, follicular lymphoma (FL) cell, chronic lymphocytic leukemia (CLL) cell, acute lymphocytic leukemia (ALL) cell, mantle cell lymphoma (MCL), or multiple myeloma.

b. Linkers

In particular embodiments, a CAR comprises a linker positioned between the extracellular domain and the transmembrane domain, optionally wherein the linker is about 2 to 100 amino acids in length. The linker can include or be composed of flexible residues such as glycine and serine so that the adjacent protein domains are free to move relative to one another. Longer linkers may be used, e.g., when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof.

In particular embodiments, the linker is P2A self-cleaving peptide, a T2A self-cleaving peptide, an E2A self-cleaving peptide, or an IgG4 hinge region polypeptide.

c. Transmembrane Domain

The transmembrane region can be any transmembrane region that can be incorporated into a functional CAR. Illustrative examples of transmembrane (TM) domains for use in CARs contemplated herein may be isolated, obtained, or derived from polypeptides including, but not limited to the alpha, beta, gamma, or delta chain of the T cell receptor, CD2, CD3, CD4, CD8α, and CD28

d. Intracellular Signaling Domain

In particular embodiments, a CAR comprises one or more intracellular signaling domains. In particular embodiments, a CAR comprises a costimulatory signaling domain and a primary signaling domain.

Illustrative examples of primary signaling domains suitable for use in CARs contemplated herein may be isolated, obtained, or derived from polypeptides including, but not limited to FcRγ, FcRβ, CD3ζ, CD79a, CD79b, and CD66d.

Illustrative examples of costimulatory domains suitable for use in CARs contemplated herein may be isolated, obtained, or derived from polypeptides including, but not limited to CD2, CD7, CD11a, CD27, CD28, CD30, CD40, CD134 (OX40), CD137 (4-1BB), CD278 (ICOS), and TNF receptor superfamily member 14 (TNFRS14).

In particular embodiments, a viral vector encodes a CAR comprising an antigen binding domain that binds CD19 or BCMA, an IgG4 linker, a CD28 derived transmembrane domain, a 4-1BB costimulatory domain, and a CD3ζ primary signaling domain.

e. DARICS

In particular embodiments, the CAR is a dimerization activated receptor initiation complex (DARIC). A DARIC provides a binding component and a signaling component that are each expressed as separate fusion proteins comprising an extracellular multimerization domain. The multimerization domains of the components associate by binding to a bridging factor (see U.S. Pat. Appl. No. 2016/0311901, which is incorporated herein by reference in its entirety). Although the bridging factor leads to formation of the DARIC system components, formation of the DARIC complex does not produce significant signaling on its own. The described DARIC complexes contemplated herein only initiate physiologically relevant signals when the DARIC binding component binds to a target antigen expressed or displayed on a target cell. In particular embodiments, a DARIC binding component comprises one or more antigen binding domains that bind to CD19 and/or CD20.

### H. Polynucleotides

The present disclosure also provides nucleic acids and polynucleotides encoding components of a multi-component cell-surface receptor or macromolecule signaling complex, transduction enhancers, and chimeric antigen receptors contemplated herein. The nucleic acid may be in the form of a construct comprising a plurality of polynucleotides encoding any of the aforementioned polypeptides. As used herein, the terms “polynucleotide,” “nucleotide,” and “nucleic acid” are intended to be synonymous with each other.

It will be understood by a skilled person that numerous different polynucleotides and nucleic acids can encode the same polypeptide as a result of the degeneracy of the genetic code. In particular embodiments, a polynucleotide comprises one or more nucleotide substitutions that do not affect the polypeptide sequence encoded by the polynucleotides contemplated herein to reflect the codon usage of any particular host organism in which the polypeptides are to be expressed.

Nucleic acids may comprise DNA or RNA, may be single-stranded or double-stranded, and may comprise synthetic or modified nucleotides. Modifications include methylphosphonate and phosphorothioate backbones, addition of acridine or poly lysine chains at the 3′ and/or 5′ ends of the molecule. In particular embodiments, polynucleotides are modified to enhance the in vivo activity or life span of polynucleotides of interest.

The terms “variant”, “homologue” or “derivative” in relation to a polynucleotide include any substitution of, variation of, modification of, replacement of, deletion of or addition of one (or more) nucleotides relative to a parent polynucleotide. A nucleic acid may produce a polypeptide which comprises one or more sequences encoding a mitogenic transduction enhancer and/or one or more sequences encoding a cytokine-based transduction enhancer. One or more polypeptides can be separated by a cleavage site or internal ribosomal entry site (IRES). The cleavage site may be self-cleaving, such that when the polypeptide is produced, it is immediately cleaved into the receptor component and the signaling component without the need for any external cleavage activity.

Various self-cleaving peptides include the Foot-and-Mouth disease virus (FMDV) 2A self-cleaving peptide and various variants and 2A-like peptides. In particular embodiments, the self-cleaving peptide has the amino acid sequence RAEGRGSLLTCGDVEENPGP (SEQ ID NO: 93) or QCTNYALLKLAGDVESNPGP (SEQ ID NO: 94).

In particular embodiments, a polynucleotide encodes a polypeptide that confers resistance to an antiangiogenic agent to the immune effector cell transduced with it.

### I. Viral Particle Tagging Proteins

In particular embodiments, a viral envelope comprises a tagging protein which comprises a binding domain which binds to a capture moiety; a spacer; and a transmembrane domain.

In particular embodiments, a tagging protein facilitates purification of a viral particle from cellular supernatant via binding of the tagging protein to the capture moiety. “Binding domain” refers to an entity, for example an epitope, which is capable of recognizing and specifically binding to a target entity, for example a capture moiety. The binding domain may comprise one or more epitopes which are capable of specifically binding to a capture moiety. For example, a binding domain may comprise at least one, two, three, four or five epitopes capable of specifically binding to a capture moiety. In particular embodiments, a binding domain comprises more than one epitope separated by a linker sequence, as described herein.

In particular embodiments, a binding domain is releasable from the capture moiety upon the addition of an entity which has a higher binding affinity for the capture moiety compared to the binding domain.

In particular embodiments, a binding domain comprises one or more streptavidin-binding epitope(s). In particular embodiments, a binding domain comprises at least one, two, three, four or five streptavidin-binding epitopes. In particular embodiments, streptavidin is a 52.8 kDa protein purified from the bacterium Streptomyces avidinii.

In particular embodiments, a binding domain comprises a biotin mimic. A “biotin mimic” refers to a short peptide sequence (for example 6 to 20, 6 to 18, 8 to 18 or 8 to 15 amino acids) which specifically binds to streptavidin. In particular embodiments, a biotin mimic may bind streptavidin with a lower binding affinity than biotin, so that biotin may be used to elute streptavidin-captured retroviral vectors. In particular embodiments, a binding domain comprises at least one, two, three, four or five biotin mimics, wherein each mimic is the same or a different mimic.

The present disclosure also provides viral particles that may be purified and methods of purification of the same. In particular embodiments, a viral envelope of a viral particle comprises a tagging protein which comprises: a binding domain which binds to a capture moiety; a spacer; and a transmembrane domain, which tagging protein facilitates purification of the viral vector from cellular supernatant via binding of the tagging protein to the capture moiety.

In particular embodiments, a binding domain of the tagging protein may comprise one or more streptavidin-binding epitope(s). In particular embodiments, a streptavidin-binding epitope(s) comprises a biotin mimic that binds streptavidin with a lower affinity than biotin, so that biotin may be used to elute streptavidin-captured retroviral vectors produced by the packaging cell. Illustrative examples of suitable biotin mimics include, but are not limited to, streptagll, flankedccstretag, and ccstreptag. In particular embodiments, a viral vector comprises a polynucleotide encoding an engineered antigen receptor. In particular embodiments, a recombinant virus is a virus-like particle (VLP).

### J. Packaging Cell Lines and Production of Viral Particles

The present disclosure also provides a host cell for the production of a recombinant retrovirus. In particular embodiments, a host cell expresses a mitogenic transduction enhancer and/or a cytokine-based transduction enhancer at the cell surface. In particular embodiments, a host cell produces a recombinant retrovirus according to the foregoing embodiments. In particular embodiments, a host cell comprises tagging proteins useful for the purification of a recombinant retrovirus.

In particular embodiments, a host cell is a packaging cell and comprises one or more genes encoding: gag, pol, an envelope polypeptide (e.g., one or more mutated viral envelope glycoproteins that retain fusogenic activity and lack cognate receptor binding activity), one or more non-viral membrane bound tropism polypeptides, and rev. In particular embodiments, a packaging cell for producing a recombinant retrovirus comprises genes encoding gag, pol, one or more non-viral membrane bound tropism polypeptides, and an envelope polypeptide (e.g., one or more mutated viral envelope glycoproteins that retain fusogenic activity and lack cognate receptor binding activity). In particular embodiments, a packaging cell for producing lentiviral particles comprises genes encoding gag, pol, an envelope polypeptide (e.g., one or more mutated viral envelope glycoproteins that retain fusogenic activity and lack cognate receptor binding activity), one or more non-viral membrane bound tropism polypeptides, and rev.

In particular embodiments, a host cell is a producer cell and comprises genes encoding gag, pol, an envelope polypeptide (e.g., one or more mutated viral envelope glycoproteins that retain fusogenic activity and lack cognate receptor binding activity), one or more non-viral membrane bound tropism polypeptides, and optionally rev and further comprises a retroviral vector or lentiviral vector. In particular embodiments, the viral vector is replication-defective, the vector is capable of integrating its genome into a target cell genome but unable to propagate itself due to a lack of structural proteins.

Packaging cells are used to propagate and isolate quantities of viral vectors, i.e., to prepare suitable titers of the retroviral vector for transduction of a target cell.

In particular embodiments, propagation and isolation comprise isolation of genes encoding a retroviral gag-pol, an envelope polypeptide (e.g., one or more mutated viral envelope glycoproteins that retain fusogenic activity and lack cognate receptor binding activity), one or more non-viral membrane bound tropism polypeptides, (and in the case of lentivirus, rev) and introduction of these genes into a host cell to produce a packaging cell line. The packaging cell line produces accessory proteins required for packaging retroviral

RNA and when a recombinant vector carrying a psi region is introduced into the packaging cell line, the accessory proteins can package the psi-positive recombinant vector to produce the recombinant viral particles.

A summary of the available packaging lines is presented in “Retroviruses” (1997 Cold Spring Harbor Laboratory Press Eds: J M Coffin, SM Hughes, HE Varmus pp 449).

Packaging cells have also been developed in which separate expression plasmids encoding gag, pol and an envelope polypeptide (e.g., one or more mutated viral envelope glycoproteins that retain fusogenic activity and lack cognate receptor binding activity), one or more non-viral membrane bound tropism polypeptides, (and, in the case of lentiviral vectors, rev) are independently transfected into a packaging cell line.

Transient transfection avoids the longer time required to generate stable vector-producing cell lines. In one embodiment, transient transfection is because the vector or retroviral packaging components are toxic to the packaging cells. Components typically used to generate retroviral/lentiviral particles include a plasmid encoding gag/pol polypeptides, a plasmid encoding an envelope polypeptide (e.g., one or more mutated viral envelope glycoproteins that retain fusogenic activity and lack cognate receptor binding activity), a plasmid encoding one or more non-viral membrane bound tropism polypeptides, and in the case of lentiviral vectors, a plasmid encoding the rev polypeptide, and a plasmid encoding the retroviral/lentiviral vector genomic RNA. In a particular embodiment, virus production involves transient transfection of one or more of these components into host cells comprising the other components required producing virus. In particular embodiments, packaging cells are selected from any mammalian cell type capable of producing retroviral/lentiviral vector particles. In particular embodiments, the packaging cells are 293T cells, or variants of 293T cells which have been adapted to grow in suspension without serum.

In particular embodiments, packaging cells are made by transient transfection with a transfer vector (a polynucleotide encoding a viral vector) and one or more expression vectors encoding gag-pol, one or more mutated viral envelope glycoproteins that retain fusogenic activity and lack cell binding activity, one or more tropism polypeptides, and optionally rev and/or one or more transduction enhancers.

In particular embodiments, a host cell or packaging cell expresses one or more tropism polypeptides and/or transduction enhancers at the cell surface.

In particular embodiments, a host cell or packaging cell comprises or expresses at the cell surface: a mitogenic transduction enhancer comprising a mitogenic domain and a transmembrane domain; and/or a cytokine-based transduction enhancer which comprises a cytokine domain and a transmembrane domain; and/or a tropism polypeptide.

In particular embodiments, a host cell may also express, at the cell surface, a tagging protein which comprises: a binding domain which binds to a capture moiety; and a transmembrane domain, which tagging protein facilitates purification of the viral particle from cellular supernatant via binding of the tagging protein to the capture moiety. In particular embodiments, a tagging protein comprises a spacer between the binding domain and the transmembrane domain.

In particular embodiments, a host cell refers to packaging cell or a producer cell.

### K. Transgenic Immune Effector Cells

The present disclosure provides a method for making an activated transgenic immune cell, in vivo or ex vivo, which comprises a step of contacting an immune effector cell with a system contemplated herein or one or more viral particles. The immune effector cells may be transduced in vivo or ex vivo. In particular embodiments, one or more recombinant retroviruses is administered to a subject to transduce an immune effector cell in vivo without any need to isolate or manipulate donor immune effector cells ex vivo. In particular embodiments, immune effector cells are manipulated ex vivo and then returned to the subject in need thereof.

Immune effector cells generally are mammalian cells, and typically are human cells, more typically primary human cells, e.g., allogeneic or autologous donor cells. The cells may be isolated from a sample, such as a biological sample, e.g., one obtained from or derived from a subject. In particular embodiments, the subject from which the cell is isolated has the disease or condition or in need of therapy or to which the therapy will be administered. In particular embodiments, a subject is a human in need of a particular therapeutic intervention, such as the adoptive cell therapy for which cells are being isolated, processed, and/or engineered. In particular embodiments, the cells are derived from the blood, bone marrow, lymph, or lymphoid organs, are cells of the immune system, such as cells of the innate or adaptive immune systems, e.g., myeloid or lymphoid cells, including lymphocytes, typically B cells, T cells, NK cells, and/or NKT cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In particular embodiments, the cells include one or more subsets of immune effector cells or other cell types including, but not limited to, whole T cell populations, CD3⁺ cells, CD4⁺ cells, CD8⁺ cells, and subpopulations thereof, including but not limited to those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

In preferred embodiments, a system comprising one or more recombinant retroviruses is administered in vivo to transduce immune effector cells including but not limited to B lymphocytes, T lymphocytes, NK cells, and/or NKT cells.

Among the sub-types and subpopulations of T cells and/or of CD4⁺ and/or of CD8⁺ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, αβ T cells, and δγ T cells.

In particular embodiments, the immune effector cells are cytotoxic T lymphocytes. A “cytotoxic T lymphocyte” (CTL) includes, but is not limited to, a T lymphocyte that expresses CD8 on the surface thereof (e.g., a CD8⁺ T cell). In particular embodiments, the immune effector cells are “memory” T cells (TM cells) that are antigen-experienced. In particular embodiments, the cell is a precursor T cell. In particular embodiments, the precursor T cell is a hematopoietic stem cell. In particular embodiments, the immune effector cell is a CD8⁺ T cytotoxic lymphocyte cell selected from the group consisting of naive CD8⁺ T cells, central memory CD8⁺ T cells, effector memory CD8⁺ T cells and bulk CD8⁺ T cells. In some embodiments, the immune effector cell is a CD4⁺ T helper lymphocyte cell that is selected from the group consisting of naive CD4⁺ T cells, central memory CD4⁺ T cells, effector memory CD4⁺ T cells, and bulk CD4⁺ T cells. As used herein, any reference to a transgenic T cell or transduced T cell, or the use thereof, may also be applied to any of the other immune effector cell types contemplated herein.

### L. Pharmaceutical Compositions and Formulations

Formulations and compositions contemplated herein comprise a system or a combination of any number of recombinant retroviruses, polypeptides, polynucleotides, small molecules and formulated in pharmaceutically acceptable or physiologically-acceptable compositions for administration to a cell, tissue, organ, or an animal, either alone, or in combination with one or more other modalities of therapy. In particular embodiments, the one or more additional pharmaceutical agents further increases transduction efficiency of a recombinant retrovirus.

In particular embodiments a composition comprises a therapeutically effective amount of a retroviral particle formulated with one or more pharmaceutically acceptable carriers, diluents, excipients or stabilizers, in the form of lyophilized formulations or aqueous solutions. In some embodiments, one or more pharmaceutically acceptable surface-active agents (surfactant), buffers, isotonicity agents, salts, amino acids, sugars, stabilizers and/or antioxidant are used in the formulation.

In some embodiments, the composition further comprises other agents, such as, e.g., cytokines, growth factors, hormones, small molecules or various pharmaceutically active agents.

Suitable pharmaceutically acceptable surfactants comprise but are not limited to polyethylen-sorbitan-fatty acid esters, polyethylene-polypropylene glycols, polyoxyethylene-stearates and sodium dodecyl sulphates. Suitable buffers comprise but are not limited to histidine-buffers, citrate-buffers, succinate-buffers, acetate-buffers and phosphate-buffers.

Isotonicity agents are used to provide an isotonic formulation. Suitable isotonicity agents comprise but are not limited to salts, including but not limited to sodium chloride (NaCl) or potassium chloride, sugars including but not limited to glucose, sucrose, trehalose or and any component from the group of amino acids, sugars, salts and combinations thereof. In some embodiments, isotonicity agents are generally used in a total amount of about 5 mM to about 350 mM.

“Pharmaceutically acceptable” refers to molecular entities and compositions that do not produce an allergic or similar untoward reaction when administered to a human.

The term “carrier” includes any and all solvents, dispersion media, vehicles, coatings, diluents, antibacterial and antifungal agents, isotonic and absorption delaying agents, buffers, carrier solutions, suspensions, colloids, and the like.

A “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media. In some embodiments, a composition comprising a carrier is suitable for parenteral administration, e.g., intravascular (intravenous or intraarterial), intraperitoneal or intramuscular administration. Pharmaceutically acceptable carriers include sterile aqueous solutions or dispersions and sterile powders for the extemporaneous preparation of sterile injectable solutions or dispersion.

The compositions may further comprise one or more polypeptides, polynucleotides, or compounds that increase the transduction efficiency, formulated in pharmaceutically acceptable or physiologically-acceptable solutions for administration to a cell or an animal, either alone, or in combination with one or more other modalities of therapy.

The pharmaceutical compositions that contain a recombinant retrovirus or retroviral particle comprising an expression cassette or vector may be in any form that is suitable for the selected mode of administration, for example, for intraventricular, intramyocardial, intracoronary, intravenous, intra-arterial, intra-renal, intraurethral, epidural, intrathecal, intraperitoneal, or intramuscular administration. The recombinant virus can be administered, as sole active agent, or in combination with other active agents, in a unit administration form, as a mixture with conventional pharmaceutical supports, to animals and human beings. In some embodiments, the pharmaceutical composition comprises cells transduced ex vivo with any of the particles contemplated herein.

In particular embodiments, a recombinant retrovirus or lentivirus, or a pharmaceutical composition comprising that retroviral particle, is effective when administered systemically (e.g., intravenously). In certain embodiments, the retroviral vectors induce expression of CAR in transduced immune cells when a recombinant retrovirus is administered systemically.

In various embodiments, the pharmaceutical compositions contain vehicles (e.g., carriers, diluents and excipients) that are pharmaceutically acceptable for a formulation capable of being injected. Exemplary excipients include a poloxamer. Formulation buffers for viral particles generally contain salts to prevent aggregation and other excipients (e.g., poloxamer) to reduce stickiness of the viral particle. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. In particular embodiments, the formulation is stable for storage and use when frozen (e.g., at less than 0° C., about −60° C., or about −72° C.). In some embodiments, the formulation is a cryopreserved solution.

In particular embodiments, compositions are formulated for in vivo administration.

The pharmaceutical compositions of the present disclosure, formulation of pharmaceutically acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intraperitoneal, and intramuscular administration and formulation. In certain embodiments, compositions contemplated herein are administered parenterally, intravenously, intramuscularly, or intraperitoneally.

A recombinant retrovirus may be used to transduce cells in vivo at an any effective dosage. In particular embodiments, a recombinant retrovirus is administered to a subject in vivo, by direct injection to the cell, tissue, organ or subject in need of therapy.

A recombinant retrovirus may also be delivered according to viral titer (TU/mL). The amount of retrovirus directly injected is determined by total TU and can vary based on both the volume that could be feasibly injected to the site and the type of tissue to be injected. In particular embodiments, the viral titer delivered is about 1×10⁹ to about 1×10¹² TU. Systemic delivery may accommodate a much larger TU, a load of about 1×10¹⁰ to about 1×10¹⁵.

In particular embodiments, a recombinant retrovirus is administered at a dose of between about 1×10¹² and 5×10¹⁵ vector genomes (vg) of the vector per kilogram (vg) of total body mass of the subject (vg/kg). In particular embodiments, a recombinant retrovirus is administered at a dose of between about 1×10¹² and 5×10¹⁵ vector particles (vp) of the vector per kilogram (vp) of total body mass of the subject (vp/kg).

Rapamune® (sirolimus, rapamycin) is available as an oral solution or tablet and is FDA approved. Per the US Prescribing Information (USPI), rapamycin is available in 1 mg/mL oral solution or 0.5, 1, or 2 mg tablets and is to be administered once daily. Rapamycin or rapalogs may also be delivered in other dosage forms and/or by other administration routes. In particular embodiments, rapamycin is administered at a dose of between about 0.1 mg/m² and 100 mg/m² of surface area of the subject. In particular embodiments, rapamycin is administered at a dose of between about 0.001 mg/m² and 100 mg/m² of surface area of the subject.

In particular embodiments, a dose of rapamycin is administered every day. In certain embodiments, a dose of rapamycin is administered about every 1, 2, 3, 4, 5, 6, 7, 8, 9, 10, 11, 12, 13, or 14 days. In some embodiments, a dose of rapamycin is administered about every 1, 2, 3, 4, 5, 6, 7, 8, 9, or 10 weeks. In other embodiments, a dose of rapamycin is administered about every 1, 2, 3, 4, 5, 6, 7, 8, 9, or 10 months.

In particular embodiments, an amount of recombinant retrovirus is administered and a first dose of rapamycin is administered about 1, 2, 3, 4, 5, 6, 7, 8, 9, 10, 11, 12, 13, or 14 days, weeks, or months post administration of the recombinant retrovirus.

In particular embodiments, administration of rapamycin or rapalog increases the number immune effector cells transduced by retroviral particles. In certain embodiments, administration of rapamycin increases the number immune effector cells transduced by retroviral particles by at least 10%, at least 20%, at least 30%, at least 40%, at least 50%, at least 60%, at least 70%, at least 80%, at least 90%, at least 1-fold, at least 2-fold, at least 3-fold, at least 5-fold, at least 7-fold, or at least 10-fold, in the subject. In particular embodiments, the increase is evaluated by the number of transduced immune effector cells 1, 2, 3, 4, 5, 6, 7, 8, 9, 10, 11, or 12 weeks or months after the first dose of the rapamycin (once the viral particle is administered), wherein the reference number is the number of transduced immune cells on the day of the first dose of rapamycin.

In particular embodiments, a recombinant retrovirus is administered via a route selected from the group consisting of parenteral, intravenous, intramuscular, subcutaneous, intratumoral, intraperitoneal, and intralymphatic. In particular embodiments, a recombinant retrovirus is administered multiple times.

In particular embodiments, a recombinant retrovirus is administered by intraperitoneal, subcutaneous, or intranodal injection.

In particular embodiments, a recombinant retrovirus is administered as a single injection. In some embodiments, the retroviral particle is administered as at least 1, at least 2, at least 3, at least 4, at least 5, at least 6, at least 7, at least 8, at least 9, or at least 10 injections.

### M. Methods of Treatment

In various embodiments, a system, recombinant retrovirus and/or composition contemplated herein are used to treat, prevent, or ameliorate at least one symptom of a disease, disorder, or condition. In particular embodiments, the disease, disorder, or condition is cancer. In particular embodiments, the cancer is a hematological malignancy or a solid tumor. In particular embodiments, the subject has relapsed or is refractory to treatment with a prior anti-cancer therapeutic.

In some embodiments, the cancer is a hematological malignancy.

In particular embodiments, a system, recombinant retrovirus and/or composition contemplated herein treats, prevents, or ameliorates at least one symptom of a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, nonHodgkin's lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

In particular embodiments, a system, recombinant retrovirus and/or composition contemplated herein treats, prevents, or ameliorates at least one symptom of a CD19-expressing B cell malignancy.

In particular embodiments, a system, recombinant retrovirus and/or composition contemplated herein treats, prevents, or ameliorates at least one symptom of a CD20-expressing B cell malignancy.

In particular embodiments, a system, recombinant retrovirus and/or composition contemplated herein treats, prevents, or ameliorates at least one symptom of a solid tumor.

In particular embodiments, a system, recombinant retrovirus and/or compositions contemplated herein treats, prevents, or ameliorates at least one symptom of prostate cancer, lung cancer, non-small cell lung cancer (NSCLC), liver cancer, cervical cancer, colon cancer, breast cancer, ovarian cancer, endometrial cancer, pancreatic cancer, melanoma, esophageal cancer, gastric cancer, stomach cancer, renal carcinoma, bladder cancer, hepatocellular carcinoma, renal cell carcinoma, urothelial carcinoma, head and neck cancer, glioma, glioblastoma, colorectal cancer, thyroid cancer, epithelial cancers, or adenocarcinomas.

In particular embodiments, a system, recombinant retrovirus and/or composition contemplated herein is administered in combination with one or more anti-cancer therapies including, but not limited to, an autologous stem cell transplant (ASCT), radiation, surgery, a chemotherapeutic agent, an immunomodulatory agent and a targeted cancer therapy.

In particular embodiments, the one or more anti-cancer therapies is selected from the group consisting of 6-mercaptopurine, abiraterone, alemtuzumab, all-trans retinoic acid, anastrozole, aprepitant, arsenic trioxide, atezolizumab, azacytidine, bafetinib, bevacizumab, bleomycin, bortezomib, bosutinib, cabazitaxel, capecitabine, carboplatin, carfilzomib, cetuximab, cisplatin, cladribine, corticosteroid, crizotinib, cyclophosphamide, cytarabine, dasatinib, daunorubicin, danusertib, decitabine, denosumab, dexamethasone, docetaxel, doxorubicin, elotozumab, eribulin, erlotinib, etoposide, everolimus, exemestane, filgrastim, fludarabine, fluorouracil, fulvestrant, gemcitabine, hydroxyurea, idarubicin, imatinib, imiquimod, ipilimumab, ixabepilone, ixazomib, lapatinib, lenalidomide, letrozole, leuprolide, melphalan, methotrexate, mitoxantrone, nilotinib, nivolumab, oxaliplatin, paclitaxel, palonosetron, pembrolizumab, pemetrexed, pomalidomide, ponatinib, prednisone, radium-223, rituximab, saracatinib, sipuleucel-T, sorafenib, sunitinib, tamoxifen, temozolomide, temsirolimus, thalidomide, tinorelbine, topotecan, tozasertib, trastuzumab, vincristine, and zoledronic acid.

All publications and patents mentioned herein are hereby incorporated herein by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

While illustrative embodiments have been illustrated and described, it will be appreciated that various changes can be made therein without departing from the spirit and scope of the invention.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A system comprising a recombinant retrovirus comprising:
(a) a viral envelope comprising (i) one or more mutated viral envelope glycoproteins that retain fusogenic activity and lack cognate receptor binding activity and (ii) a non-viral membrane-bound tropism polypeptide, and
(b) a recombinant retroviral vector comprising at least a first polynucleotide and a second polynucleotide, each polynucleotide comprising a polynucleotide sequence encoding a polypeptide component of a macromolecular complex, wherein assembly of the macromolecular complex in a cell transduced with the first and second polynucleotides promotes growth and/or survival of a cell.

2. A system comprising
(a) a first recombinant retrovirus comprising:
(i) a viral envelope comprising one or more mutated viral envelope glycoproteins that retain fusogenic activity and lack cognate receptor binding activity and a non-viral membrane-bound tropism polypeptide, and
(ii) a recombinant retroviral vector comprising a first polynucleotide encoding a polypeptide component of a macromolecular complex; and
(b) a second recombinant retrovirus comprising:
(i) a viral envelope comprising one or more mutated viral envelope glycoproteins that retain fusogenic activity and lack cognate receptor binding activity and a non-viral membrane-bound tropism polypeptide, and
(ii) a recombinant retroviral vector comprising a second polynucleotide encoding a polypeptide component of a macromolecular complex;
wherein assembly of the macromolecular complex in the cell transduced with the first polynucleotide and the second polynucleotide promotes growth and/or survival of a cell.

3. The system of claim 1 or claim 2, wherein the macromolecular complex is a multi-component cell-surface receptor.

4. The system of claim 1, wherein the recombinant retrovirus is a recombinant lentivirus.

5. The system of claim 2, wherein the recombinant retroviruses are recombinant lentiviruses.

6. The system of any one of claims 1 to 5, wherein the recombinant retrovirus is a recombinant lentivirus selected from the group consisting of: human immunodeficiency virus 1 (HIV-1); human immunodeficiency virus 2 (HIV-2), visna-maedi virus (VMV); caprine arthritis-encephalitis virus (CAEV); equine infectious anemia virus (EIAV); feline immunodeficiency virus (FIV); bovine immune deficiency virus (BIV); and simian immunodeficiency virus (SIV).

7. The system of any one of claims 1 to 6, wherein assembly of the macromolecular complex is controlled by a ligand.

8. The system of any one of claims 1 to 7, wherein the one or more mutated viral envelope glycoproteins comprise a vesiculovirus envelope glycoprotein, one or more morbillivirus envelope glycoproteins or one or more henipavirus envelope glycoproteins.

9. The system of claim 8, wherein the vesiculovirus is selected from the group consisting of: vesicular stomatitis Alagoas virus (VSAV; Alagoas vesiculovirus), Carajás virus (CJSV; Carajas vesiculovirus), Chandipura virus (CHPV; Chandipura vesiculovirus), Cocal virus (COCV; Cocal vesiculovirus), vesicular stomatitis Indiana virus (VSIV; Indiana vesiculovirus), Isfahan virus (ISFV; Isfahan vesiculovirus), Maraba virus (MARAV; Maraba vesiculovirus), Morreton virus (MORV; Morreton vesiculovirus), vesicular stomatitis New Jersey virus (VSNJV; New Jersey vesiculovirus), and Piry virus (PIRYV; Piry vesiculovirus).

10. The system of claim 8 or claim 9, wherein the vesiculovirus envelope glycoprotein is a vesiculovirus G protein.

11. The system of any one of claims 8 to 10, wherein the vesiculovirus G protein is a COCV G glycoprotein (COCV-G) or a VSIV G glycoprotein (VSIV-G).

12. The system of claim 11, wherein the VSIV-G envelope protein comprises one or more of:
(a) one or more amino acid substitutions at H8, N9, Q10, K47, K50, A51, S183, S179, N180, 1182, M184, Y209, 1347, T350, T352, E353, and R354;
(b) an insertion of TT between N9 and Q10, an insertion of GGS between H8 and N9, an insertion of GGS between N9 and Q10, an insertion of TT between N208 and Y209, an insertion of GGS between P46 and K47, and an insertion of GGS between N208 and Y209;
(c) amino acid substitutions at K47 and/or R354; or
(d) a deletion of residues 1-8.

13. The system of claim 11 or claim 12, wherein the VSIV-G envelope protein comprises one or more amino acid substitutions at H8, K47, Y209, and R354.

14. The system of any one of claims 11 to 13, wherein the VSIV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q.

15. The system of any one of claims 11 to 14, wherein the VSIV-G envelope protein comprises the amino acid sequence set forth in SEQ ID NO: 2, wherein X₁=I, X₂=A, X₃=Q, and X₄=A; X₁=I, X₂=A, X₃=Q, and X₄=G; X₁=I, X₂=A, X₃=Q, and X₄=F; X₁=I, X₂=A, X₃=Q, and X₄=Q; X₁=L, X₂=A, X₃=Q, and X₄=A; X₁=L, X₂=A, X₃=Q, and X₄=G; X₁=L, X₂=A, X₃=Q, and X₄=F; X₁=L, X₂=A, X₃=Q, and X₄=Q; X₁=I, X₂=A, X₃=H, and X₄=A; X₁=I, X₂=A, X₃=H, and X₄=G; X₁=I, X₂=A, X₃=H, and X₄=F; X₁=I, X₂=A, X₃=H, and X₄=Q; X₁=L, X₂=A, X₃=H, and X₄=A; X₁=L, X₂=A, X₃=H, and X₄=G; X₁=L, X₂=A, X₃=H, and X₄=F; X₁=L, X₂=A, X₃=H, and X₄=Q; X₁=I, X₂=G, X₃=Q, and X₄=A; X₁=I, X₂=G, X₃=Q, and X₄=G; X₁=I, X₂=G, X₃=Q, and X₄=F; X₁=I, X₂=G, X₃=Q, and X₄=Q; X₁=L, X₂=G, X₃=Q, and X₄=A; X₁=L, X₂=G, X₃=Q, and X₄=G; X₁=L, X₂=G, X₃=Q, and X₄=F; X₁=L, X₂=G, X₃=Q, and X₄=Q; X₁=I, X₂=G, X₃=H, and X₄=A; X₁=I, X₂=G, X₃=H, and X₄=G; X₁=I, X₂=G, X₃=H, and X₄=F; X₁=I, X₂=G, X₃=H, and X₄=Q; X₁=L, X₂=G, X₃=H, and X₄=A; X₁=L, X₂=G, X₃=H, and X₄=G; X₁=L, X₂=G, X₃=H, and X₄=F; X₁=L, X₂=G, X₃=H, and X₄=Q; X₁=I, X₂=F, X₃=Q, and X₄=A; X₁=I, X₂=F, X₃=Q, and X₄=G; X₁=I, X₂=F, X₃=Q, and X₄=F; X₁=I, X₂=F, X₃=Q, and X₄=Q; X₁=L, X₂=F, X₃=Q, and X₄=A; X₁=L, X₂=F, X₃=Q, and X₄=G; X₁=L, X₂=F, X₃=Q, and X₄=F; X₁=L, X₂=F, X₃=Q, and X₄=Q; X₁=I, X₂=F, X₃=H, and X₄=A; X₁=I, X₂=F, X₃=H, and X₄=G; X₁=I, X₂=F, X₃=H, and X₄=F; X₁=I, X₂=F, X₃=H, and X₄=Q; X₁=L, X₂=F, X₃=H, and X₄=A; X₁=L, X₂=F, X₃=H, and X₄=G; X₁=L, X₂=F, X₃=H, and X₄=F; X₁=L, X₂=F, X₃=H, and X₄=Q; X₁=I, X₂=Q, X₃=Q, and X₄=A; X₁=I, X₂=Q, X₃=Q, and X₄=G; X₁=I, X₂=Q, X₃=Q, and X₄=F; X₁=I, X₂=Q, X₃=Q, and X₄=Q; X₁=L, X₂=Q, X₃=Q, and X₄=A; X₁=L, X₂=Q, X₃=Q, and X₄=G; X₁=L, X₂=Q, X₃=Q, and X₄=F; X₁=L, X₂=Q, X₃=Q, and X₄=Q; X₁=I, X₂=Q, X₃=H, and X₄=A; X₁=I, X₂=Q, X₃=H, and X₄=G; X₁=I, X₂=Q, X₃=H, and X₄=F; X₁=I, X₂=Q, X₃=H, and X₄=Q; X₁=L, X₂=Q, X₃=H, and X₄=A; X₁=L, X₂=Q, X₃=H, and X₄=G; X₁=L, X₂=Q, X₃=H, and X₄=F; and X₁=L, X₂=Q, X₃=H, and X₄=Q.

16. The system of any one of claims 8 to 10, wherein the vesiculovirus G protein is COCV-G.

17. The system of claim 16, wherein the COCV-G envelope protein comprises one or more amino acid substitutions at K47 and R354.

18. The system of claim 16 or claim 17, wherein the COCV-G envelope protein comprises one or more amino acid substitutions selected from the group consisting of: K47A and R354A; K47A and R354G; K47A and R354F; K47A and R354Q; K47G and R354A; K47G and R354G; K47G and R354F; K47G and R354Q; K47F and R354A; K47F and R354G; K47F and R354F; K47F and R354Q; K47Q and R354A; K47Q and R354G; K47Q and R354F; and K47Q and R354Q.

19. The system of any one of claims 16 to 18, wherein the COCV-G envelope protein comprises the amino acid sequence set forth in SEQ ID NO: 4, wherein X₁=A and X₂=A; X₁=A and X₂=G; X₁=A and X₂=F; X₁=A and X₂=Q; X₁=G and X₂=A; X₁=G and X₂=G; X₁=G and X₂=F; X₁=G and X₂=Q; X₁=F and X₂=A; X₁=F and X₂=G; X₁=F and X₂=F; X₁=F and X₂=Q; X₁=Q and X₂=A; X₁=Q and X₂=G; X₁=Q and X₂=F; or X₁=Q and X₂=Q.

20. The system of claim 8, wherein the one or more morbillivirus envelope glycoproteins are measles virus F (MV-F) and measles virus H (MV-H).

21. The system of claim 20, wherein the MV-H protein comprises one or more amino acid substitutions at Y481, R533, S548, and F549.

22. The system of claim 20 or claim 21, wherein the MV-H protein comprises one or more amino acid substitutions selected from the group consisting of: Y481A, R533A, S548L, and F549S.

23. The system of claim 8, wherein the one or more henipavirus envelope glycoproteins are nipah virus F (NiV-F) and nipah virus G (NiV-G).

24. The system of claim 23, wherein the NiV-G protein comprises one or more amino acid substitutions at E501, W504, Q530, and E533.

25. The system of claim 23 or claim 24, wherein the NiV-G protein comprises one or more amino acid substitutions at E501A, W504A, Q530A, and E533A.

26. The system of any one of claims 1 to 25, wherein the non-viral membrane-bound tropism polypeptide comprises an extracellular antigen targeting domain, a spacer polypeptide, and a transmembrane domain.

27. The system of claim 26, wherein the extracellular antigen targeting domain binds an antigen expressed on an immune effector cell.

28. The system of claim 26 or claim 27, wherein the extracellular antigen targeting domain binds an antigen expressed on an immune effector cell selected from the group consisting of: the alpha, beta, gamma, or delta chain of the T cell receptor, CD2, CD3δ, CD3ε CD3γ, CD4, CD8α, and CD8β.

29. The system of any one of claims 26 to 28, wherein the extracellular antigen targeting domain comprises an antibody or antigen binding fragment thereof that binds an antigen expressed on the immune effector cell.

30. The system of any one of claims 26 to 29, wherein the extracellular antigen targeting domain comprises an anti-CD3 antibody or antigen binding fragment selected from the group consisting of OKT3, UCHT1, YTH12.5, TR66, and variants thereof, and antibodies and antigen binding fragments that have at least 80%, at least 85%, at least 90%, at least 95%, at least 99%, or 100% identity thereto.

31. The system of any one of claims 1 to 30, wherein the system comprises a first polynucleotide comprising a polynucleotide sequence encoding a first polypeptide component of the macromolecular complex comprising an FKBP-rapamycin complex binding (FRB) polypeptide or a functional variant thereof, and a second polynucleotide comprising a polynucleotide sequence encoding a second polypeptide component of the macromolecular complex comprising an FK506 binding protein (FKBP) polypeptide or a functional variant thereof; and/or wherein the ligand is rapamycin.

32. The system of any one of claims 1 to 31, wherein expression of the macromolecular complex is under the control of an inducible genetic or biochemical system.

33. The system of any one of claims 1 to 32, wherein each polynucleotide is operatively linked to a promoter.

34. The system of claim 33, wherein the promoter is an inducible promoter.

35. The system of any one of claims 1 to 34, wherein at least one of the polynucleotides comprises a polynucleotide sequence that confers resistance to an immunosuppressive agent.

36. The system of claim 35, wherein the polynucleotide sequence that confers resistance to an immunosuppressive agent encodes a polypeptide that binds rapamycin, wherein optionally, the polypeptide is FRB.

37. The system of any one of claims 1 to 36, wherein the recombinant retrovirus or recombinant retroviruses are capable of transducing T cells, NK cells, or NKT cells in vivo or ex vivo.

38. The system of any one of claims 1 to 37, wherein the viral envelope further comprises one or more transduction enhancers, wherein the transduction enhancer is selected from the group consisting of a T cell activation receptor, a NK-cell activation receptor, and a co-stimulatory molecule.

39. The system of claim 38, wherein the one or more transduction enhancers comprise one or more of CD80, CD86, CD137L, OX40L, and ICOSL.

40. The system of any one of claims 1 to 39, wherein the first polynucleotide comprises a polynucleotide sequence encoding: a promoter, an FKBP polypeptide, an IL-2 receptor transmembrane domain, an IL2Rγ intracellular signaling domain; and a first chimeric antigen receptor (CAR).

41. The system of any one of claims 1 to 40, wherein the second polynucleotide comprises a polynucleotide sequence encoding: a promoter, an FRB polypeptide, an IL-2 receptor transmembrane domain, an IL2Rβ intracellular signaling domain; and a second CAR.

42. The system of claim 40 or claim 41, wherein the FKBP polypeptide and FRB polypeptide heterodimerize in the presence of rapamycin to promote growth and/or survival of a cell.

43. The system of any one of claims 1 to 42, wherein the promoter is MND.

44. The system of any one of claims 41 to 43, wherein the first CAR is an anti-CD19 CAR, an anti-CD22 CAR, an anti-CD20 CAR, an anti-CD79A CAR, an anti-CD79b CAR, an anti-CD38 CAR, an anti-GPCR5D CAR, or an anti-BCMA CAR.

45. The system of any one of claims 41 to 44, wherein the second CAR is different from the first CAR and is selected from the group consisting of: an anti-CD19 CAR, an anti-CD22 CAR, an anti-CD20 CAR, an anti-CD79A CAR, an anti-CD79b CAR, an anti-CD38 CAR, an anti-GPCR5D CAR, or an anti-BCMA CAR.

46. A method, comprising: administering to a subject the system of any of claims 1 to 45.
